(19) United States
(12) Patent Application Publication
(10) Pub. No.: US 2013/0053407 A1
(43) Pub. Date: Feb. 28, 2013

(54) PYRROLO [3,2-D] PYRIMIDIN-3-YL DERIVATIVES USED AS ACTIVATORS OF AMPK

(71) Applicants: Olivier Mirguet, Les Ulis (FR); Anne Marie Jeanne Bouillot, Le Ulis (FR)

(72) Inventors: Olivier Mirguet, Les Ulis (FR); Anne Marie Jeanne Bouillot, Le Ulis (FR)

(73) Assignee: GlaxoSmithKline LLC, Philadelphia, PA (US)

(21) Appl. No.: 13/695,970

(22) PCT Filed: May 3, 2011
(86) PCT No.: PCT/EP2011/057013

§ 371 Date: Nov. 2, 2012

Related U.S. Application Data
(60) Provisional application No. 61/331,496, filed on May 5, 2010.

(51) Int. Cl. A61K 31/519; A61P 35/00; C07D 487/04

(52) U.S. Cl. 514/265.1; 544/280

(57) ABSTRACT

Pyrrolopyrimidones compounds of the formula (I), salts thereof, and pharmaceutical compositions containing them are disclosed herein, as well as methods for their use in medicine, for instance as activators of AMPK.

### FIELD OF THE INVENTION

The present invention relates to a novel class of compounds which are activators of AMP-activated protein kinase (AMPK) (AMPK-activators), compositions comprising said compounds, methods of synthesis and uses for such compounds in treating and/or preventing various diseases mediated by AMPK, such as diabetes, metabolic syndrome, atherosclerosis, dyslipidaemia, obesity, hypertension, cerebral ischemia, cognitive defect and cancer.

### BACKGROUND OF THE INVENTION

AMPK has been established as a sensor and regulator of cellular energy homeostasis (Hardie, D. G. and Hawley, S. A. AMP-activated protein kinase: the energy charge hypothesis revisited. Bioessays 23: 1112 (2001), Kemp, B. E. et. al. AMP-activated protein kinase, super metabolic regulator. Biochem. Soc. Transactions 31:162 (2003)). Allosteric activation of this kinase due to rising AMP levels occurs in states of cellular energy depletion. The resulting serine/threonine phosphorylation of target enzymes leads to an adaptation of cellular metabolism to the low energy state. The net effect of AMPK activation induced changes is inhibition of ATP consuming processes and activation of ATP generating pathways, and therefore regeneration of ATP stores. Examples of AMPK substrates include acetyl-CoA-carboxylase (ACC) and HMG-CoA-reductase (Carling, D. et. al. A common bicyclic protein kinase cascade inactivates the regulatory enzymes of fatty acid and cholesterol biosynthesis. FEBS Letters 223:217 (1987)). Phosphorylation and therefore inhibition of ACC leads to a decrease in fatty acid synthesis (ATP-consuming) and at the same time to an increase in fatty acid oxidation (ATP-generating). Phosphorylation and resulting inhibition of HMG-CoA reductase leads to a decrease in cholesterol synthesis. Other substrates of AMPK include hormone sensitive lipase (Garton, A. J. et. al. Phosphorylation of bovine hormone-sensitive lipase by the AMP-activated protein kinase. A possible antilipolytic mechanism. Eur. J. Biochem. 179:249 (1989)), glycerol-3-phosphate acyltransferase (Muoio, D. M. et. al. AMP-activated kinase reciprocally regulates triacylglycerol synthesis and fatty acid oxidation in liver and muscle: evidence that sn-glycerol-3-phosphate acyltransferase is a novel target. Biochem. J. 338:783 (1999)), malonyl-CoA decarboxylase (Saha, A. K. et. al. Activation of malonyl-CoA decarboxylase in rat skeletal muscle by contraction and the AMP-activated protein kinase activator 5-aminoimidazole-4-carboxamide-1-.beta.-D-ribofuranoside. J. Biol. Chem. 275:24279 (2000)), some of which are potential drug targets for components of the metabolic syndrome. Additional processes that are believed to be regulated through AMPK activation, but for which the exact AMPK substrates have not been identified, include stimulation of glucose transport in skeletal muscle and expressional regulation of key genes in fatty acid and glucose metabolism in liver (Hardie, D. G. and Hawley, S. A. AMP-activated protein kinase: the energy charge hypothesis revisited. Bioessays 23: 1112 (2001), Kemp, B. E. et. al. AMP-activated protein kinase, super metabolic regulator. Biochem. Soc. Transactions 31:162 (2003), Musi, N. and Goodyear, L. J. Targeting the AMP-activated protein kinase for the treatment of Type II diabetes. Current Drug Targets-Immune, Endocrine and Metabolic Disorders 2:119 (2002)). For example, decreased expression of glucose-6-phosphatase (Lochhead, P. A. et. al. 5-aminoimidazole-4-carboxamide riboside mimics the effects of insulin on the expression of the 2 key gluconeogenic genes PEPCK and glucose-6-phosphatase. Diabetes 49:896 (2000)), a key enzyme in hepatic glucose production, and SREBP-1c (Zhou, G. et. al. Role of AMP-activated protein kinase in mechanism of metformin action. The J. of Clin. Invest. 108: 1167 (2001)), a key lipogenic transcription factor, has been found following AMPK stimulation.

More recently an involvement of AMPK in the regulation of not only cellular but also whole body energy metabolism has become apparent. It was shown that the adipocyte-derived hormone leptin leads to a stimulation of AMPK and therefore to an increase in fatty acid oxidation in skeletal muscle (Minokoshi, Y. et. al. Leptin stimulates fatty-acid oxidation by activating AMP-activated protein kinase. Nature 415: 339 (2002)). Adiponectin, another adipocyte derived hormone leading to improved carbohydrate and lipid metabolism, has been demonstrated to stimulate AMPK in liver and skeletal muscle (Yamauchi, T. et. al. Adiponectin stimulates glucose utilization and fatty acid oxidation by activating AMP-activated protein kinase. Nature Medicine 8: 1288 (2002), Tomas, E. et. al. Enhanced muscle fat oxidation and glucose transport by ACRP30 globular domain: Acetyl-CoA carboxylase inhibition and AMP-activated protein kinase activation. PNAS 99: 16309 (2002)). The activation of AMPK in these circumstances seems to be independent of increasing cellular AMP levels but rather due to phosphorylation by one or more yet to be identified upstream kinases.

Based on the knowledge of the above-mentioned consequences of AMPK activation, certain beneficial effects could be expected from in vivo activation of AMPK. In liver, decreased expression of gluconeogenic enzymes could reduce hepatic glucose output and improve overall glucose homeostasis, and both direct inhibition and/or reduced expression of key enzymes in lipid metabolism could lead to decreased fatty acid and cholesterol synthesis and increased fatty acid oxidation. Stimulation of AMPK in skeletal muscle could increase glucose uptake and fatty acid oxidation with resulting improvement of glucose homeostasis and, due to a reduction in intra-myocyte triglyceride accumulation, to improved insulin action. Finally, the increase in energy expenditure could lead to a decrease in body weight. The combination of these effects in the metabolic syndrome could be expected to reduce the risk for acquiring cardiovascular diseases.

Several studies in rodents support this hypothesis (Bergeron, R. et. al. Effect of 5-aminoimidazole-4-darboxamide-1(beta)-D-ribofuranoside infusion on in vivo glucose metabolism in lean and obese Zucker rats. Diabetes 50:1076 (2001), Song, S. M. et. al. 5-Aminoimidazole-4-carboxamide ribonucleoside treatment improves glucose homeostasis in insulin-resistant diabetic (ob/ob) mice. Diabetologia 45:56 (2002), Halseth, A. E. et. al. Acute and chronic treatment of ob/ob and db/db mice with AICAR decreases blood glucose concentrations. Biochem. and Biophys. Res. Comm. 294:798 (2002), Buhl, E. S. et. al. Long-term AICAR administration reduces metabolic disturbances and lowers blood pressure in rats displaying features of the insulin resistance syndrome. Diabetes 51: 2199 (2002)). Until recently most in vivo studies have relied on the AMPK activator AICAR, a cell permeable precursor of ZMP. ZMP acts as an intracellular AMP mimic, and, when accumulated to high enough levels, is able to stimulate AMPK activity (Corton, J. M. et. al. 5-Aminoimidazole-4-carboxamide ribonucleoside, a specific method for activating AMP-activated protein kinase in intact cells. Eur. J. Biochem. 229: 558 (1995)). However, ZMP also acts as an AMP mimic in the regulation of other enzymes, and is therefore not a specific AMPK activator (Musi, N. and Goodyear, L. J. Targeting the AMP-activated protein kinase for the treatment of Type II diabetes. Current Drug Targets-Immune, Endocrine and Metabolic Disorders 2:119 (2002)). Several in vivo studies have demonstrated beneficial effects of both acute and chronic AICAR administration in rodent models of obesity and Type II diabetes (Bergeron, R. et. al. Effect of 5-aminoimidazole-4-carboxamide-1(beta)-D-ribofuranoside infusion on in vivo glucose metabolism in lean and obese Zucker rats. Diabetes 50:1076 (2001), Song, S. M. et. al. 5-Aminoimidazole-4-carboxamide ribonucleoside treatment improves glucose homeostasis in insulin-resistant diabetic (ob/ob) mice. Diabetologia 45:56 (2002), Halseth, A. E. et. al. Acute and chronic treatment of ob/ob and db/db mice with AICAR decreases blood glucose concentrations. Biochem. and Biophys. Res. Comm. 294:798 (2002), Buhl, E. S. et. al. Long-term AICAR administration reduces metabolic disturbances and lowers blood pressure in rats displaying feature of the insulin resistance syndrome. Diabetes 51: 2199 (2002)). For example, 7 week AICAR administration in the obese Zucker (fa/fa) rat leads to a reduction in plasma triglycerides and free fatty acids, an increase in HDL cholesterol, and a normalization of glucose metabolism as assessed by an oral glucose tolerance test (Minokoshi, Y. et. al. Leptin stimulates fatty-acid oxidation by activating AMP-activated protein kinase. Nature 415: 339 (2002)). In both ob/ob and db/db mice, 8 day AICAR administration reduces blood glucose by 35% (Halseth, A. E. et. al. Acute and chronic treatment of ob/ob and db/db mice with AICAR decreases blood glucose concentrations. Biochem. and Biophys. Res. Comm. 294:798 (2002)). In addition to AICAR, more recently it was found that the diabetes drug metformin can activate AMPK in vivo at high concentrations (Zhou, G. et. al. Role of AMP-activated protein kinase in mechanism of metformin action. The J. of Clin. Invest. 108: 1167 (2001), Musi, N. et. al. Metformin increases AMP-activated protein kinase activity in skeletal muscle of subjects with Type II diabetes. Diabetes 51: 2074 (2002)), although it has to be determined to what extent its antidiabetic action relies on this activation. As with leptin and adiponectin, the stimulatory effect of metformin is indirect via a mild inhibition of mitochondrial respiratory chain complex 1 (Leverve X. M. et al. Mitochondrial metabolism and type-2 diabetes: a specific target of metformin. Diabetes Metab. 29: 6588 (2003)). In addition to pharmacologic intervention, several transgenic mouse models have been developed in the last years, and initial results are becoming available. Expression of dominant negative AMPK in skeletal muscle of transgenic mice has demonstrated that the AICAR effect on stimulation of glucose transport is dependent on AMPK activation (Mu, J. et. al. A role for AMP-activated protein kinase in contraction and hypoxia-regulated glucose transport in skeletal muscle. Molecular Cell 7: 1085 (2001)), and therefore likely not caused by non-specific ZMP effects. Similar studies in other tissues will help to further define the consequences of AMPK activation. It is believed that pharmacologic activation of AMPK may have benefits in relation to metabolic syndrome with improved glucose and lipid metabolism and a reduction in body weight. To qualify a patient as having metabolic syndrome, three out of the five following criteria must be met: elevated blood pressure above 130/85 mmHg, fasting blood glucose above 110 mg/dl, abdominal obesity above 40″ (men) or 35″ (women) waist circumference, and blood lipid changes as defined by an increase in triglycerides above 150 mg/dl or decreased HDL cholesterol below 40 mg/dl (men) or 50 mg/dl (women). Therefore, the combined effects that may be achieved through activation of AMPK in a patient who qualifies as having metabolic syndrome would raise the interest of this target.

Lowering of blood pressure has been reported to be a consequence of AMPK activation (Buhl, E. S. et. al. Long-term AICAR administration reduces metabolic disturbances and lowers blood pressure in rats displaying feature of the insulin resistance syndrome. Diabetes 51: 2199 (2002)), therefore activation of AMPK might have beneficial effects in hypertension. Through combination of some or all of the above-mentioned effects stimulation of AMPK may to reduce the incidence of cardiovascular diseases (e.g. MI, stroke). Increased fatty acid synthesis is a characteristic of many tumor cells, therefore decreased synthesis of fatty acids through activation of AMPK could be useful as a cancer therapy (Huang X. et al. Important role of the LKB1-AMPK pathway in suppressing tumorigenesis in PTEN-deficient mice. Biochem J. 412: 211 (2008). Stimulation of AMPK has been shown to stimulate production of ketone bodies from astrocytes (Blazquez, C. et. al. The AMP-activated protein kinase is involved in the regulation of ketone body production by astrocytes. J. Neurochem. 73: 1674 (1999)), and might therefore be a strategy to treat ischemic events in the brain. Stimulation of AMPK has been shown to improve cognition and neurodegenerative diseases in a mice modele (Dagon Y. et al. Nutritional status, cognition, and survival: a new role for leptin and AMP kinase. J. Biol. Chem. 280:42142 (2005)). Stimulation of AMPK has been shown to stimulate expression of uncoupling protein 3 (UCP3) in skeletal muscle (Zhou, M. et. al. UCP-3 expression in skeletal muscle: effects of exercise, hypoxia, and AMP-activated protein kinase. Am. J. Physiol. Endocrinol. Metab. 279: E622 (2000)) and might therefore be a way to prevent damage from reactive oxygen species. Endothelial NO synthase (eNOS) has been shown to be activated through AMPK mediated phosphorylation (Chen, Z.-P., et. al. AMP-activated protein kinase phosphorylation of endothelial NO synthase. FEBS Letters 443: 285 (1999)), therefore AMPK activation may be used to improve local circulatory systems.

### SUMMARY OF THE INVENTION

The present invention provides a compound of formula (I):

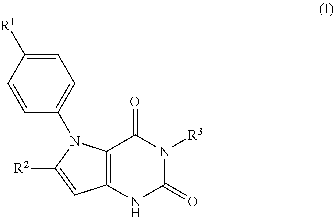

wherein

R¹ represents —C₆₋₁₀aryl substituted by an —OH group and optionally further substituted by one or two groups independently selected from:
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —CO₂H;
(vi) —C₁₋₄haloalkyl;
(vii) —OC₁₋₄haloalkyl;
(viii) —XC(═O)C₁₋₄alkyl; or
(ix) halogen;

R² represents H or halogen;

R³ represents

(a)
(i) —C₁₋₄alkyl wherein the alkyl group is substituted by one or two groups
independently selected from: —OH or —CO₂H;
(ii) H;
(iii) —C₁₋₄haloalkyl;
(iv) —C₁₋₄alkyleneOC₁₋₄alkyl; or
(v) —C₁₋₄alkylene(═O)XC₁₋₄alkyl; or
(b) —C₆₋₁₀aryl, wherein the —C₆₋₁₀aryl is unsubstituted or substituted by one or two groups independently selected from:
(i) —C₁₋₄alkyl wherein the alkyl group is unsubstituted or substituted by one or two groups independently selected from: —OH or —CO₂H,
(ii) —C₁₋₄alkoxy;
(iii) —OH;
(iv) —CN;
(v) —NO₂;
(vi) —CO₂H;
(vii) —C₁₋₄haloalkyl;
(viii) —OC₁₋₄haloalkyl;
(ix) —C₁₋₄alkylene(═O)XC₁₋₄alkyl; or
(x) halogen;

X represents O or —NR; and

R⁴ represents H or —C₁₋₄alkyl;

or a salt thereof.

In another aspect, the present invention provides pharmaceutical compositions comprising a compound of formula (I) or a pharmaceutically acceptable salt thereof.

In another aspect, the present invention provides methods of treating diabetes, metabolic syndrome, atherosclerosis, dyslipidaemia, obesity, hypertension, cerebral ischemia, cognitive defect and cancer comprising administration of a therapeutically effective amount of a compound of formula (I) or a pharmaceutically acceptable salt thereof to a subject in need thereof.

In another aspect, the invention provides a compound of formula (I) or a pharmaceutically acceptable salt thereof for use in human or veterinary medical therapy.

In another aspect, the invention provides a compound of formula (I) or a pharmaceutically acceptable salt thereof, for use in the treatment or prophylaxis of diabetes, metabolic syndrome, atherosclerosis, dyslipidaemia, obesity, hypertension, cerebral ischemia, cognitive defect and cancer.

In another aspect, the invention provides the use of a compound of formula (I) or a pharmaceutically acceptable salt thereof in the manufacture of a medicament for the treatment or prophylaxis of diabetes, metabolic syndrome, atherosclerosis, dyslipidaemia, obesity, hypertension, cerebral ischemia, cognitive defect and cancer.

### DESCRIPTION OF THE EMBODIMENTS

All aspects and embodiments of the invention described herein are in respect of compounds of formula I, unless otherwise specified.

In one aspect of the invention, R¹ represents phenyl substituted by an —OH group and optionally further substituted by one or two groups independently selected from:
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —XC(═O)C₁₋₄alkyl; or
(ix) halogen.

In one embodiment, the phenyl group is substituted at the 2-position by an —OH group.

In another aspect of the invention, R¹ represents —C₆₋₁₀aryl substituted by an —OH group and optionally further substituted by a group independently selected from:
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —XC(═O)C₁₋₄alkyl; or
(ix) halogen.

In another aspect of the invention, R¹ represents phenyl substituted by an —OH group and optionally further substituted by a group independently selected from:
(i) —C₁₋₄alkyl wherein the alkyl group is unsubstituted or substituted by one or two independently selected from: —OH or —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —XC(═O)C₁₋₄alkyl; or
(ix) halogen.

In one embodiment, the phenyl group is substituted at the 2-position by an —OH group.

In another aspect of the invention, R¹ represents —C₆₋₁₀aryl substituted by an —OH group and further substituted by a group independently selected from:
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —XC(═O)C₁₋₄alkyl; or
(ix) halogen.

In another aspect of the invention, R¹ represents phenyl substituted by an —OH group and further substituted by a group independently selected from:
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —XC(═O)C₁₋₄alkyl; or
(ix) halogen.

In one embodiment, the phenyl group is substituted at the 2-position by an —OH group.

In another aspect of the invention, R¹ represents phenyl substituted by an —OH group and further substituted by a halogen. In one embodiment, the phenyl group is substituted at the 2-position by an —OH group.

In another aspect of the invention, R¹ represents phenyl substituted by an —OH group and further substituted by fluoro. In one embodiment, the phenyl group is substituted at the 2-position by an —OH group.

In another aspect of the invention, R¹ represents phenyl substituted by an —OH group and further substituted by chloro. In one embodiment, the phenyl group is substituted at the 2-position by an —OH group.

In another aspect of the invention, R¹ represents phenyl substituted by an —OH group and further substituted by —C₁₋₄alkyl. In one embodiment, the phenyl group is substituted at the 2-position by an —OH group.

In another aspect of the invention, R¹ represents phenyl substituted by an —OH group and further substituted by —CH₃ (methyl). In one embodiment, the phenyl group is substituted at the 2-position by an —OH group.

In another aspect of the invention, R¹ represents phenyl substituted by an —OH group and further substituted by —C₁₋₄alkoxy. In one embodiment, the phenyl group is substituted at the 2-position by an —OH group.

In another aspect of the invention, R¹ represents phenyl substituted by an —OH group and further substituted by —OCH₃ (methoxy). In one embodiment, the phenyl group is substituted at the 2-position by an —OH group.

In another aspect of the invention, R¹ represents phenyl substituted by an —OH group. In one embodiment, the phenyl group is substituted at the 2-position by an —OH group.

In another aspect of the invention, R¹ represents

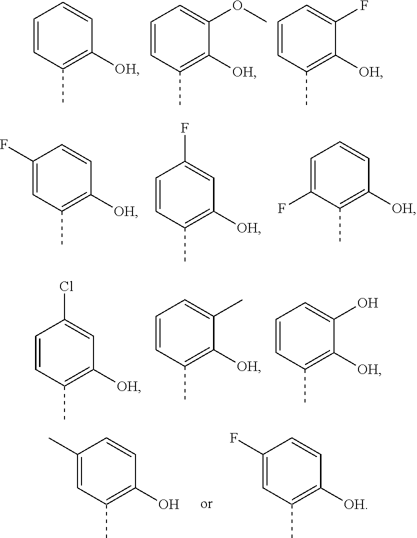

In a further aspect of the invention, R¹ represents

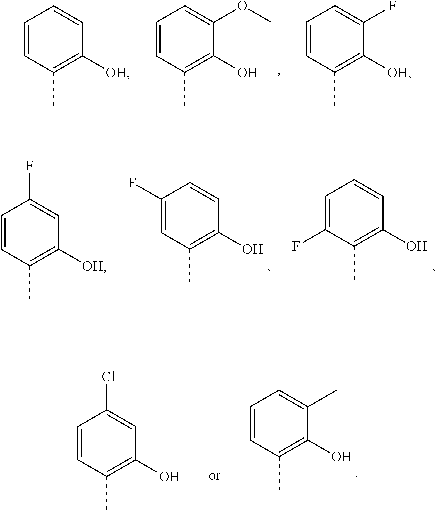

In another aspect of the invention, R¹ represents

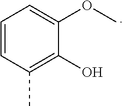

In one aspect of the invention R² represents H or chloro.

In one aspect of the invention, R² represents H.

In another aspect of the invention, R² presents halogen. In a further aspect, R² represent chloro.

In one aspect of the invention, R³ represents:

(a)
(i) —C₁₋₄alkyl wherein the alkyl group is substituted by one or two groups independently selected from: —OH or —CO₂H;
(ii) H;
(iii) —C₁₋₄haloalkyl;
(iv) —C₁₋₄alkyleneOC₁₋₄alkyl; or
(v) —C₁₋₄alkylene(═O)XC₁₋₄alkyl.

In a further aspect of the invention, R³ represents:

(a)
(i) —C₁₋₄alkyl wherein the alkyl group is substituted by one or two groups independently selected from: —OH or —CO₂H;
(ii) H;
(iii) —C₁₋₄alkyleneOC₁₋₄alkyl; or
(iv) —C₁₋₄alkylene(═O)XC₁₋₄alkyl.

In another aspect of the invention, R³ represents —C₁₋₄alkyl wherein the alkyl group is substituted by one or two substituents independently selected from: —OH or —CO₂H.

In another aspect of the invention, R³ represents —C₁₋₄alkyl wherein the alkyl group is substituted by a group independently selected from: —OH or —CO₂H.

In another aspect of the invention, R³ represents —(CH₂)₂OH, —(CH₂)₃OH, —(CH₂)₃CO₂H, —CH(CH₃)CO₂H, CH₂CO₂H or —(CH₂)₂CO₂H.

In another aspect of the invention, R³ represents —(CH₂)₂OH, —(CH₂)₂OCH₃ or —(CH₂)₂CO₂H.

In another aspect of the invention, R³ represents H.

In another aspect of the invention, R³ represents —C₁₋₄haloalkyl.

In another aspect of the invention, R³ represents —C₁₋₄alkyleneOC₁₋₄alkyl.

In one embodiment, R³ represents (CH₂)₂OCH₃ or (CH₂)₃OCH₃.

In another aspect of the invention, R³ represents —C₁₋₄alkylene(═O)XC₁₋₄alkyl.

In another aspect of the invention, R³ represents —(CH₂)₂CO₂Et, —(CH₂)₂CO₂ⁱPr, —CH₂CO₂ⁱPr, —CH₂CO₂Et, or —(CH₂)₂C(O)NHMe.

In another aspect of the invention, R³ represents —C₆₋₁₀aryl, wherein the —C₆₋₁₀aryl is unsubstituted or substituted by one or two groups independently selected from:

(b)
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —NO₂;
(vi) —CO₂H;
(vii) —C₁₋₄haloalkyl;
(viii) —OC₁₋₄haloalkyl;
(ix) —C₁₋₄alkylene(═O)XC₁₋₄alkyl; or
(x) halogen.

In another aspect of the invention, R³ represents phenyl unsubstituted or substituted by one or two groups independently selected from:

(b)
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —NO₂;
(vi) —CO₂H;
(vii) —C₁₋₄haloalkyl;
(viii) —OC₁₋₄haloalkyl;
(ix) —C₁₋₄alkylene(═O)XC₁₋₄alkyl; or
(x) halogen.

In another aspect of the invention, R³ represents phenyl unsubstituted or substituted by a group independently selected from:

(b)
(i) —C₁₋₄alkyl wherein the alkyl group is unsubstituted or substituted by one or two groups independently selected from: —OH or —CO₂H;
(ii) —C₁₋₄alkoxy;
(iii) —OH;
(iv) —CN;
(v) —NO₂;
(vi) —CO₂H;
(vii) —C₁₋₄haloalkyl;
(viii) —OC₁₋₄haloalkyl;
(ix) —C₁₋₄alkylene(═O)XC₁₋₄alkyl; or
(x) halogen.

In another aspect of the invention, R³ represents phenyl unsubstituted or substituted by one or two groups independently selected from —CH₃, —OCH₃, —OH, —CF₃, —OCF₃, —CN, —NO₂, CO₂H and halogen. In a further aspect R³ represents phenyl unsubstituted or substituted by one or two groups independently selected from —CH₃, —OCH₃, —OH, —CF₃, —OCF₃, —CN, —NO₂, CO₂H, Cl and F.

In another aspect of the invention, R³ represents phenyl unsubstituted or substituted by one or two groups independently selected from —CH₃, —OCH₃, —OH, —CF₃, —OCF₃, —CN, —NO₂ or halogen.

In another aspect of the invention, R³ represents phenyl unsubstituted or substituted by one or two groups independently selected from —CH₃, —OCH₃, —CF₃, —CN, —NO₂, CO₂H and halogen. In a further aspect, R³ represents phenyl unsubstituted or substituted by one or two groups independently selected from —CH₃, —OCH₃, —CF₃, —CN, —NO₂, CO₂H, Cl and F.

In another aspect of the invention, R³ represents phenyl unsubstituted or substituted by a group independently selected from —CH₃, —OCH₃, —OH, —CF₃, —OCF₃, —CN, —NO₂ or halogen. In a further aspect of the invention, R³ represents phenyl unsubstituted or substituted by a group independently selected from —CH₃, —OCH₃, —OH, —CF₃, —OCF₃, —CN, —NO₂, F and Cl.

In another aspect of the invention, R³ represents phenyl substituted by one or two groups independently selected from —CH₃, —OCH₃, —OH, —CF₃, —OCF₃, —CN, —NO₂ or halogen. In a further aspect of the invention, R³ represents phenyl substituted by one or two groups independently selected from —CH₃, —OCH₃, —OH, —CF₃, —OCF₃, —CN, —NO₂, F and Cl.

In another aspect of the invention, R³ represents phenyl substituted by a group independently selected from —CH₃, —OCH₃, —OH, —CF₃, —OCF₃, —CN, —NO₂ or halogen. In a further aspect of the invention, R³ represents phenyl substituted by a group independently selected from —CH₃, —OCH₃, —OH, —CF₃, —OCF₃, —CN, —NO₂, Cl and F.

In one aspect of the invention, R³ represents naphthyl.

In one aspect of the invention, X represents O. In another aspect of the invention, X represents —NR⁴.

In one aspect of the invention R⁴ represents H. In another aspect of the invention R⁴ represents —C₁₋₄alkyl. In another aspect of the invention R⁴ represents —CH₃ (methyl).

In one aspect of the invention there is provided a compound of formula (I) as hereinbefore defined, provided that the compound of formula (I) is not 4-[6-chloro-5-(2′-hydroxy-3′-methyl-4-biphenylyl)-2,4-dioxo-1,2,4,5-tetrahydro-3H-pyrrolo[3,2-d]pyrimidin-3-yl]benzonitrile, 6-chloro-3-(3-fluorophenyl)-5-[2′-hydroxy-3′-(methyloxy)-4-biphenylyl]-1H-pyrrolo[3,2-d]pyrimidine-2,4(3H,5H)-dione or 6-chloro-5-(4′-chloro-2′-hydroxy-4-biphenylyl)-1H-pyrrolo[3,2-d]pyrimidine-2,4(3H,5H)-dione.

Each of the aspects of the invention are independent unless stated otherwise. Nevertheless the skilled person will understand that all the permutations of the aspects herein described are within the scope of the invention. Thus it is to be understood that the present invention covers all combinations of suitable, convenient and exemplified aspects described herein.

As used herein, the term “alkyl” refers to a straight or branched saturated hydrocarbon chain containing the specified number of carbon atoms. For example, —C₁₋₄alkyl refers to a straight or branched alkyl containing at least 1, and at most 4, carbon atoms. Examples of “alkyl” as used herein include, but are not limited to, methyl, ethyl, n-propyl, n-butyl, isobutyl, isopropyl and t-butyl.

As used herein, the term “alkoxy group”’ refers to straight or branched O-alkyl group, wherein alkyl is as defined hereinabove. Examples of “alkoxy” as used herein include, but are not limited to methoxy, ethoxy, butoxy and but-2-oxy.

As used herein, the term “alkylene” refers to a straight or branched chain saturated hydrocarbon linker group containing the specified number of carbon atoms. For example, —C₁₋₄alkylene refers to a straight or branched chain saturated hydrocarbon linker group containing at least 1, and at most 4, carbon atoms. Examples of “alkylene” as used herein include, but are not limited to, methylene (—CH₂—), ethylene (—CH₂CH₂—) and iso-propylene (—C(CH₃)₂—).

As used herein, the term “—C₆₋₁₀aryl” refers to an aromatic carbocyclic moiety containing 6 to 10 carbon ring-atoms. The term includes both monocyclic and bicyclic ring systems and bicyclic structures at least a portion of which is aromatic and the other part is saturated, partially or fully unsaturated. Examples of aryl groups as used herein include, but are not limited to, naphthyl, anthryl, phenanthryl, indanyl, indenyl, azulenyl, azulanyl, fluorenyl and phenyl; and more specifically phenyl.

As used herein, the term “halogen” or “halo” refers to a fluorine (fluoro), chlorine (chloro), bromine (bromo) or iodine (iodo) atom.

As used herein, the term “haloalkyl” refers to an alkyl group having the specified number of carbon atoms and wherein at least one hydrogen atom is replaced with a halogen atom, for example a fluoro atom. For example, —C₁₋₄haloalkyl refers to an alkyl group containing at least 1, and at most 4, carbon atoms and at least one halogen atom, for example a fluoro atom. Examples of “haloalkyl” groups used herein include, but are not limited to, trifluoromethyl (—CF₃).

As used herein, the term “substituted” refers to substitution with the named substituent or substituents, multiple degrees of substitution being allowed unless otherwise stated.

For the avoidance of doubt, the term “independently” means that where more than one substituent is selected from a number of possible substituents, those substituents may be the same or different.

Certain compounds of formula (I) are capable of forming base addition salts.

Salts of compounds of formula (I) which are suitable for use in medicine are those wherein the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts.

Also included in the present invention are pharmaceutically acceptable salt complexes. In certain embodiments of the invention, pharmaceutically acceptable salts of the compounds according to formula I may be preferred over the respective free base or free acid because such salts impart greater stability or solubility to the molecule thereby facilitating formulation into a dosage form. Therefore, the present invention also covers the pharmaceutically acceptable salts of the compounds of formula (I).

Therefore, in one aspect of the invention there is provided a compound of formula (I) or a salt thereof wherein the salt is a pharmaceutically acceptable salt.

As used herein, the term “pharmaceutically acceptable”, refers to salts, molecular entities and other ingredients of compositions that are generally physiologically tolerable and do not typically produce untoward reactions when administered to a subject (e.g. human). The term “pharmaceutically acceptable” also means approved by a regulatory agency of the Federal or a state government or listed in the U.S. Pharmacopoeia or other generally recognized pharmacopoeia for use in a subject, and more particularly in humans.

As used herein, the term “subject” refers to an animal, in particular a mammal and more particularly to a human or a domestic animal or an animal serving as a model for a disease (e.g. mouse, monkey, etc.). In one aspect, the subject is a human.

Suitable pharmaceutically acceptable salts will be apparent to those skilled in the art and include for example base addition salts (e.g. ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine). For a review on suitable salts see Berge et al. J. Pharm. Sci., 1977, 66, 1-19. The invention includes within its scope all possible stoichiometric and non-stoichiometric forms of the salts of the compounds of formula (I).

Those skilled in the art of organic chemistry will appreciate that many organic compounds can form complexes with solvents in which they are reacted or from which they are precipitated or crystallized. These complexes are known as “solvates”.

Certain compounds of formula (I) or salts thereof may form solvates.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Most preferably the solvent used is water and the solvate may also be referred to as a hydrate.

Solvates of compounds of formula (I) which are suitable for use in medicine are those wherein the solvent is pharmaceutically acceptable. However, solvates having non-pharmaceutically acceptable solvents are within the scope of the present invention, for example, for use as intermediates in the preparation of other compounds of formula (I) and their pharmaceutically acceptable salts.

As used herein, the term “compounds of the invention” means the compounds according to formula (I) and pharmaceutically acceptable salts thereof. The term “a compound of the invention” means any one of the compounds of the invention as defined below.

Prodrugs of the compounds of formula (I) are included within the scope of the present invention. In one aspect, the invention only comprises compounds having the structure represented by formula (I).

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987 and in D. Fleishner, S. Ramon and H. Barba “Improved oral drug delivery: solubility limitations overcome by the use of prodrugs”, Advanced Drug Delivery Reviews (1996) 19(2) 115-130. Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein hydroxy, amine or carboxylic acid groups are bonded to any group that, when administered to a patient, cleaves to form the hydroxy, amine or carboxylic acid groups. Thus, representative examples of prodrugs include (but are not limited to) phosphonate, carbamate, acetate, formate and benzoate derivatives of hydroxy, amine or carboxylic acid functional groups of the compounds of formula (I).

Certain compounds of formula (I) may exist in stereoisomeric forms (e.g. they may contain one or more asymmetric carbon atoms). The individual stereoisomers (enantiomers and diastereomers) and mixtures or racemic mixtures thereof are included within the scope of the present invention. The invention also extends to conformational isomers of compounds of formula (I). Likewise, it is understood that compounds of formula (I) may exist in tautomeric forms other than that shown in the formula and these are also included within the scope of the present invention.

It will be appreciated that racemic compounds of formula (I) may be optionally resolved into their individual enantiomers. Such resolutions may conveniently be accomplished by standard methods known in the art. For example, a racemic compound of formula (I) may be resolved by chiral preparative HPLC. An individual stereoisomer may also be prepared from a corresponding optically pure intermediate or by resolution, such as H.P.L.C. of the corresponding mixture using a suitable chiral support or by fractional crystallisation of the diastereoisomeric salts formed by reaction of the corresponding mixture with a suitable optically active acid or base, as appropriate.

In one aspect, the present invention comprises a compound of formula (I) selected from the group consisting of Examples 1 to 67 or a salt thereof.

In a further aspect, the present invention comprises a compound of formula (I) selected from the group consisting of Examples 1-3, 7-33 and 47-61 or a salt thereof.

Compounds of the invention have been found to activate AMPK and may therefore be useful in the treatment of diabetes, metabolic syndrome, atherosclerosis, dyslipidaemia, obesity, hypertension, cerebral ischemia, cognitive defect and cancer.

Within the context of the present invention, the terms describing the indications used herein are classified in the Merck Manual of Diagnosis and Therapy, 17^{th} Edition and/or the International Classification of Diseases 10^{th} Edition (ICD-10). The various subtypes of the disorders mentioned herein are contemplated as part of the present invention.

In one aspect, the invention provides a compound of formula (I) or a pharmaceutically acceptable salt thereof for use in medical therapy.

In one aspect, the invention provides the use of a compound of formula (I) or a pharmaceutically acceptable salt thereof for the manufacture of a medicament for the treatment or prophylaxis of a disease or a condition mediated by AMPK activation

In one aspect, the invention provides the use of a compound of formula (I) or a pharmaceutically acceptable salt thereof for the manufacture of a medicament for the treatment of a disease or a condition mediated by AMPK activation

In another aspect, the invention provides the use of a compound of formula (I) or a pharmaceutically acceptable salt thereof for the manufacture of a medicament for the treatment or prophylaxis of diabetes, metabolic syndrome, atherosclerosis, dyslipidaemia, obesity, hypertension, cerebral ischemia, cognitive defect and cancer.

In another aspect, the invention provides the use of a compound of formula (I) or a pharmaceutically acceptable salt thereof for the manufacture of a medicament for the treatment of diabetes, metabolic syndrome, atherosclerosis, dyslipidaemia, obesity, hypertension, cerebral ischemia, cognitive defect and cancer. In a further aspect the disease or condition is selected from the group consisting of diabetes, metabolic syndrome, atherosclerosis, dyslipidaemia, obesity, hypertension, cerebral ischemia, cognitive defect and cancer.

In another aspect, the invention provides the use of a compound of formula (I) or a pharmaceutically acceptable salt thereof for the manufacture of a medicament for the treatment or prophylaxis of Type II diabetes, dyslipidaemia and cancer. In another aspect, the invention provides the use of a compound of formula (I) or a pharmaceutically acceptable salt thereof for the manufacture of a medicament for the treatment of Type II diabetes, dyslipidaemia and cancer. In one embodiment, the disorder is either Type II diabetes, dyslipidemia or cancer.

In one aspect, the invention provides a compound of formula (I) or a pharmaceutically acceptable salt thereof for use in the treatment or prophylaxis of a disease or a condition mediated by AMPK activation

In another aspect, the invention provides a compound of formula (I) or a pharmaceutically acceptable salt thereof for use in the treatment or prophylaxis of diabetes, metabolic syndrome, atherosclerosis, dyslipidaemia, obesity, hypertension, cerebral ischemia, cognitive defect and cancer. In a more particular aspect, the invention provides a compound of formula (I) or a pharmaceutically acceptable salt thereof for use in the treatment of diabetes, metabolic syndrome, atherosclerosis, dyslipidaemia, obesity, hypertension, cerebral ischemia, cognitive defect or cancer. In a further aspect the disease or condition is selected from the group consisting of diabetes, metabolic syndrome, atherosclerosis, dyslipidaemia, obesity, hypertension, cerebral ischemia, cognitive defect and cancer

In another aspect, the invention provides a compound of formula (I) or a pharmaceutically acceptable salt thereof for use in the treatment or prophylaxis of Type II diabetes, dyslipidaemia and cancer.

In another aspect, the invention provides a compound of formula (I) or a pharmaceutically acceptable salt thereof for use in the treatment of Type II diabetes, dyslipidaemia and cancer. In one embodiment, the disease is either Type II diabetes, dyslipidemia or cancer.

In one aspect, the invention provides a method for the treatment or prophylaxis of a disease or a condition susceptible to amelioration by an AMPK activator in a subject in need thereof comprising administering to said subject a therapeutically effective amount of a compound of formula (I) or pharmaceutically acceptable salt thereof. In one embodiment, the invention provides a method for the treatment of a disease or a condition susceptible to amelioration by an AMPK activator in a subject in need thereof comprising administering to said subject a therapeutically effective amount of a compound of formula (I) or pharmaceutically acceptable salt thereof.

In another aspect, the invention provides a method for the treatment or prophylaxis of diabetes, metabolic syndrome, atherosclerosis, dyslipidaemia, obesity, hypertension, cerebral ischemia, cognitive defect and cancer in a subject in need thereof comprising administering to said subject a therapeutically effective amount of a compound of formula (I) or pharmaceutically acceptable salt thereof. In another aspect, the invention provides a method for the treatment of diabetes, metabolic syndrome, atherosclerosis, dyslipidaemia, obesity, hypertension, cerebral ischemia, cognitive defect and cancer in a subject in need thereof comprising administering to said subject a therapeutically effective amount of a compound of formula (I) or pharmaceutically acceptable salt thereof. In a further aspect the disease or condition is selected from the group consisting of diabetes, metabolic syndrome, atherosclerosis, dyslipidaemia, obesity, hypertension, cerebral ischemia, cognitive defect and cancer.

In another aspect, the invention provides a method for the treatment or prophylaxis of Type II diabetes, dyslipidaemia and cancer in a subject in need thereof comprising administering to said subject a therapeutically effective amount of a compound of formula (I) or pharmaceutically acceptable salt thereof. In another aspect, the invention provides a method for the treatment of Type II diabetes, dyslipidaemia and cancer in a subject in need thereof comprising administering to said subject a therapeutically effective amount of a compound of formula (I) or pharmaceutically acceptable salt thereof. In one embodiment, the disease is Type II diabetes, dyslipidaemia or cancer.

It will be appreciated that reference to “treatment” and “therapy” refers to acute treatment.

It will be appreciated that reference to prophylaxis refers to retardation of progression of the disease, and may include the suppression of symptom recurrence in an asymptomatic patient.

### Pharmaceutical Compositions

While it is possible that, for use in the methods of the invention, a compound of formula (I) or a pharmaceutically acceptable salt thereof may be administered as the bulk substance, it is preferable to present the active ingredient in a pharmaceutical formulation, for example, wherein the agent is in admixture with at least one pharmaceutically acceptable carrier selected with regard to the intended route of administration and standard pharmaceutical practice.

Accordingly, the present invention also includes a pharmaceutical composition comprising a) a compound of formula (I) or a pharmaceutically acceptable salt thereof and b) one or more pharmaceutically acceptable carriers.

The term “pharmaceutically acceptable carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers or diluents are well known in the pharmaceutical art, and are described, for example, in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s) and/or coating agent(s).

The carrier, diluent and/or excipient must be “pharmaceutically acceptable” in the sense of being compatible with the other ingredients of the composition and not deleterious to the recipient thereof.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use.

Examples of pharmaceutically acceptable diluent(s) useful in the compositions of the invention include, but are not limited to water, ethanol, propylene glycol and glycerine.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of pharmaceutically acceptable suspending agents useful in the compositions of the invention include, but are not limited tosorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non aqueous vehicles (which may include edible oils), for example almond oil, oily esters such as glycerine, propylene glycol, or ethyl alcohol; preservatives, for example methyl or propyl p hydroxybenzoate or sorbic acid.

Examples of pharmaceutically acceptable coating materials useful in the compositions of the invention include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof.

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

The present invention relates to a pharmaceutical composition for the treatment or prophylaxis of Type II diabetes, dyslipidaemia or cancer comprising a compound of formula (I) or a pharmaceutically acceptable salt thereof. In one embodiment, the present invention relates to a pharmaceutical composition for the treatment of Type II diabetes, dyslipidaemia or cancer comprising a compound of formula (I) or a pharmaceutically acceptable salt thereof.

The present invention further relates to a pharmaceutical composition comprising a) 10 to 2000 mg of a compound of formula (I) or a pharmaceutically acceptable salt thereof, and b) 0.1 to 2 g of one or more pharmaceutically acceptable carriers.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any suitable route, and include those in a form adapted for oral, parenteral, transdermal, inhalation, sublingual, topical, implant, nasal, enterally (or other mucosally) administration to mammals including humans. The pharmaceutical compositions may be formulated in conventional manner using one or more pharmaceutically acceptable carriers or excipients. In one aspect, the pharmaceutical composition is formulated for oral administration

The compositions may be in the form of tablets, capsules, powders, granules, lozenges, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

The compounds of the invention may also, for example, be formulated as suppositories containing conventional suppository bases e.g. cocoa butter or other glyceride for use in human or veterinary medicine or as pessaries e.g., containing conventional pessary bases.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (HFA 134AT) or 1,1,1,2,3,3,3-heptafluoropropane (HFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant e.g. sorbitan trioleate.

Capsules and cartridges (made, for example, from gelatin) for use in an inhaler or insufflator may be formulated to contain a powder mix of the compound and a suitable powder base such as lactose or starch.

Advantageously, agents such as a local anaesthetic, preservative and buffering agent can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compounds of the invention may be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved, for example, by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to colour the film. Suppositories are be prepared by using carriers like cocoa butter, suppository bases such as Suppocire C, and Suppocire NA50 (supplied by Gattefossé Deutschland GmbH, D-Weil am Rhein, Germany) and other Suppocire type excipients obtained by interesterification of hydrogenated palm oil and palm kernel oil (C₈-C₁₈ triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

The compositions may contain from 0.1% by weight, preferably from 10-60% by weight, of the active ingredient, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

The compounds of formula (I) or pharmaceutically acceptable salt(s) thereof may also be used in combination with other therapeutic agents. The invention thus provides, in a further aspect, a combination comprising a) a compound of formula (I) or pharmaceutically acceptable salt thereof and b) one or more further therapeutically active agent(s).

The combinations referred to above may conveniently be presented for use in the form of a pharmaceutical composition and thus pharmaceutical compositions comprising a combination as defined above together with one or more pharmaceutically acceptable carriers thereof represent a further aspect of the invention.

Compounds of the invention may be administered in combination with other therapeutically active agents. Preferred therapeutic agents are selected from the list: bisguanidine, metformin, a DPP-IV inhibitor, sitagliptin, an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA: cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor.

When the compound of formula (I) or pharmaceutically acceptable salt thereof is used in combination with a second therapeutically active agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention.

The individual components of such combinations may be administered either sequentially or simultaneously in separate or combined pharmaceutical formulations by any convenient route.

When administration is sequential, either the AMPK activator or the second therapeutically active agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

### Methods of Preparation

Compounds of formula (I) and salts thereof may be prepared by the general methods outlined hereinafter or any method known in the art, said methods constituting a further aspect of the invention. R¹ to R⁴ and X are as defined above unless otherwise specified. Throughout the specification, general formulae are designated by Roman numerals (I), (II), (III), (IV) etc.

In a general process, compounds of formula (I) or salts thereof (other than those wherein R¹ and/or R³ contain an ester group) may be prepared according to reaction scheme 1 by reacting compounds of formula (II) or salts thereof, wherein P₁ is a suitable protecting group such as alkyl (i.e. ethyl), in the presence of an inorganic base such as sodium or sodium hydroxide in a suitable solvent such as ethanol (suitably at 80 to 90° C.).

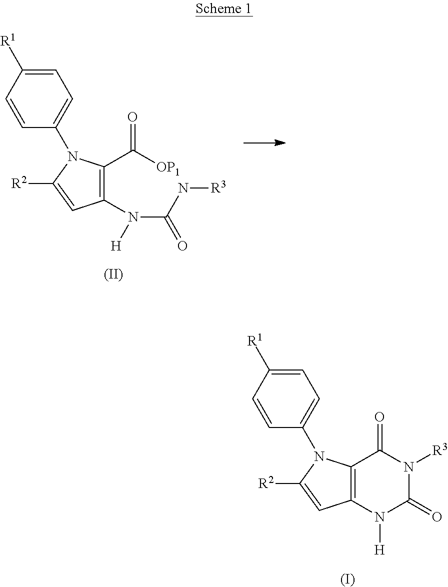

Compounds of formula (II) or salts thereof may be prepared according to reaction scheme 2 by reacting compounds of formula (III) or salts thereof, wherein P₁ is a suitable protecting group such as alkyl (i.e. ethyl), with the appropriate isocyanate (IV) in the presence in a suitable solvent such as xylene or toluene (suitably at 80 to 160° C.).

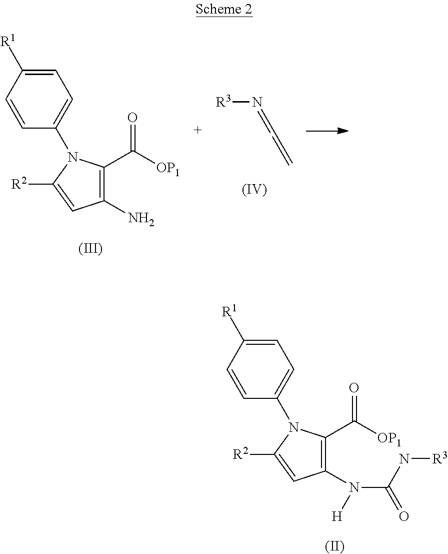

Compounds of formula (IV) are commercially available or may be prepared by methods known in the literature or processes known to those skilled in the art.

Compounds of formula (II) or salts thereof, may be also prepared according to reaction scheme 3 by reacting compounds of formula (III) or salts thereof wherein P₁ is a suitable protecting group such as alkyl (i.e. ethyl), with the appropriate amine (V) in the presence of carbonyl diimidazole in a suitable solvent such as dichloromethane at RT with subsequent heating or in the presence of triphosgene using a suitable base such as triethylamine in dichloromethane at RT with subsequent heating.

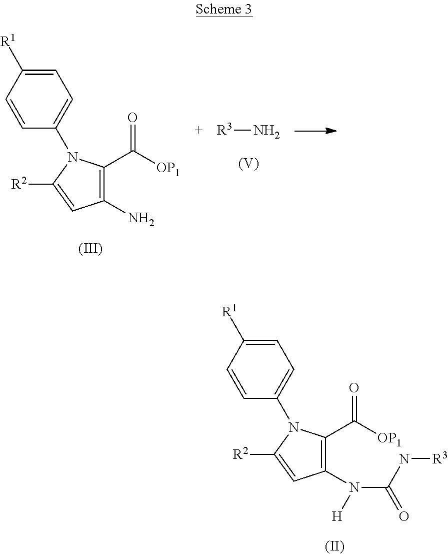

Compounds of formula (V) are commercially available or may be prepared by methods known in the literature or processes known to those skilled in the art.

Compounds of formula (II) or salts thereof may also be prepared according to reaction scheme 3a by reacting compounds of formula (III) or salts thereof wherein P₁ is a suitable protecting group such as alkyl (i.e. ethyl), with the appropriate carboxylic acid (XVIII) in the presence of diphenyl azidophosphate using a suitable base such as triethylamine in toluene at 90° C. Diphenyl azoduiphosphate is reacted with the carboxylic acid (XVIII) at 50° C. before subsequent reaction with compounds of formula (III) or salts thereof at 90° C.

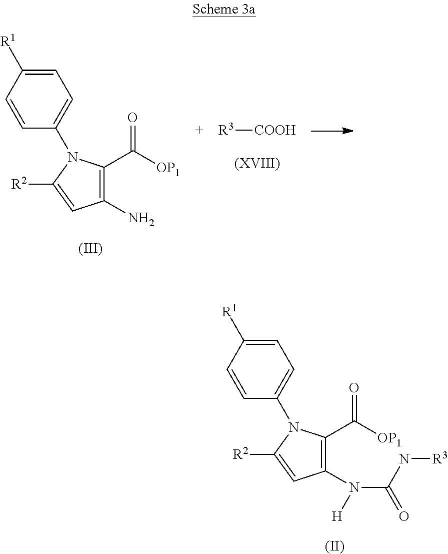

Compounds of formula (XVIII) are commercially available or may be prepared by methods known in the literature or by processes known to those skilled in the art.

Compounds of formula (III) or salts thereof may be prepared according Scheme 4 reacting the compounds of formula (VI) or salts thereof, wherein P₁ is a suitable protecting group such as alkyl (i.e. ethyl) and L₁ is a suitable leaving group such as bromo or iodo, with the appropriate aryl-boronic acid (VII) in the presence of an inorganic base such as cesium carbonate and a catalyst (such as Pd(Ph₃P)₄) in a suitable solvent such as 1,4-dioxane at reflux. Althernatively the compound of formula (VII) may be replaced with the appropriate dioxoborolane compound.

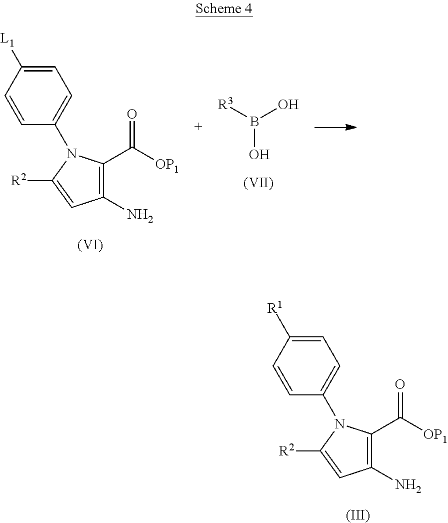

Compounds of formula (VII) are commercially available or may be prepared by methods known in the literature or processes known to those skilled in the art.

Compounds of formula (VI) or salts thereof may be prepared according to reaction scheme 5 by deprotection of a compound of formula (VIII) or salts thereof, wherein P₁ is a suitable protecting group such as alkyl (i.e. ethyl), P₂ is a suitable protecting group such as acetyl and L₁ is a suitable leaving group such as bromo or iodo. Where P₂ is acetyl, this step comprises reacting compounds of formula (VIII) in the presence of an acid such as HCl in a suitable solvent such as ethanol (suitably at reflux).

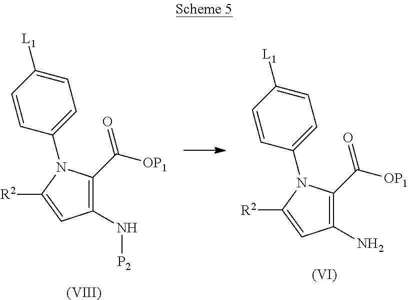

Compounds of formula (VIII) or salts thereof may be prepared according to reaction scheme 6 by reacting compounds of formula (IX) or salts thereof, wherein P₁ is a suitable protecting group such as alkyl (i.e. ethyl), P₂ is a suitable protecting group such as acetyl, with the appropriate boronic acid (X) wherein L₁ is a suitable leaving group such as bromo or iodo in the presence of a copper catalyst such as copper (II) acetate and a base such as pyridine or triethylamine in a suitable solvent such as DCM (suitably at RT).

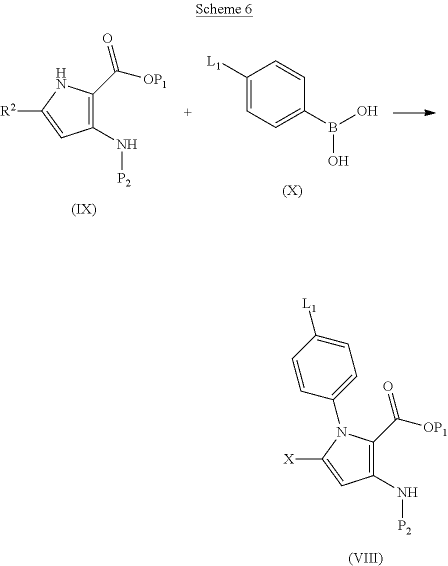

Compounds of formula (X) are commercially available or may be prepared by methods known in the literature or processes known to those skilled in the art.

Compounds of formula (IX), wherein R² is halo (formula (IXa)), may be prepared according to reaction scheme 7 by reacting a compound of formula (IX), wherein R² is H (formula IXb)), with the appropriate N-halo-succinimide (XI), in a suitable solvent such as chloroform or THF (suitably between room temperature and 35° C.), wherein P₁ is a suitable protecting group such as alkyl (i.e. ethyl), P₂ is a suitable protecting group such as acetyl and X is halo.

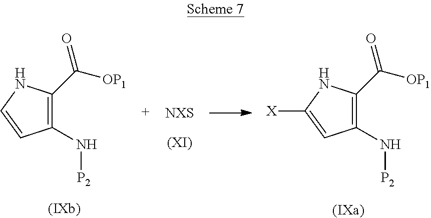

Compounds of formula (XI) are commercially available or may be prepared by methods known in the literature or processes known to those skilled in the art.

Compounds of formula (IXb) may be prepared according to Scheme 7a by reacting compounds of formula (XII) or a salt thereof, wherein P₁ is a suitable protecting group such as alkyl (i.e. ethyl), with a suitable protecting group derivative P₂—X (XIII) wherein X is halo and P₂ is a suitable protecting group such as acetyl (for instance acetyl chloride) in the presence of an amine at 0° C. to RT.

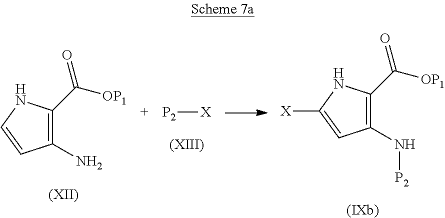

Compounds of formula (XII) and (XIII) are either commercially available or may be prepared by methods known in the literature or processes known to those skilled in the art.

Alternatively, compounds of formula (I) or salts thereof may also be prepared according to reaction scheme 8 by reacting compounds of formula (XIV) or salts thereof wherein L₁ is a suitable leaving group such as bromo or iodo with the appropriate aryl-boronic acid (VII) in the presence of an inorganic base such as cesium carbonate and a catalyst (such as Pd(Ph₃P)₄) in a suitable solvent such as 1,4-dioxane or 1,4-dioxane and water (suitably at 80 to 160° C.).

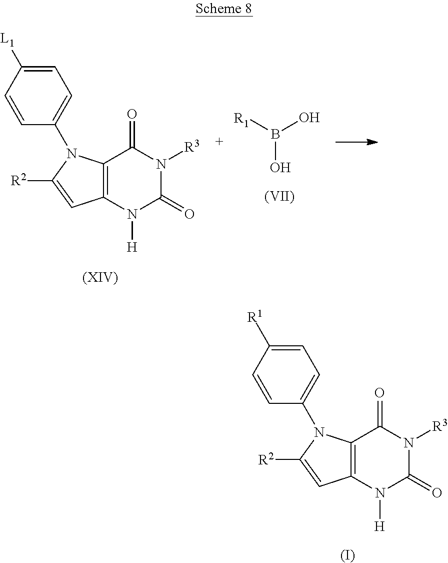

Alternatively the compound of formula (VII) can be replaced with the appropriate dioxoborolane compound.

Compounds of formula (XIV) may be prepared from compounds of formula (VIII) by following the analogous method described in reaction scheme 2 followed by reaction scheme 1. Alternatively, compounds of formula (XIV), wherein R³ represents hydrogen, can be prepared by treatment of compounds of formula (VIII) with urea at elevated temperature (e.g. 250° C.).

Alternatively, compounds of formula (III) or salts thereof may be prepared according to reaction scheme 9 by reacting compounds of formula (XVII) or salts thereof, wherein P₁ and P₂ are as hereinbefore defined, in the presence of an acid such as HCl in a suitable solvent such as ethanol (suitably at reflux).

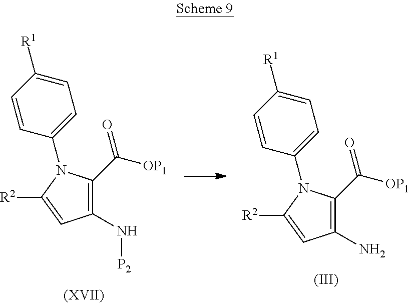

Compounds of formula (XVII) or salts thereof may be prepared according Scheme 10 reacting the compounds of formula (VIII) or salts thereof, wherein P₁, P₂ and L₁ are as hereinbefore defined, with the appropriate aryl-boronic acid (VII) in the presence of an inorganic base such as cesium carbonate and a catalyst (such as Pd(Ph₃P)₄) in a suitable solvent such as 1,4-dioxane at reflux. Alternatively the compound of formula (VII) can be replaced with the corresponding dioxoborolane compound.

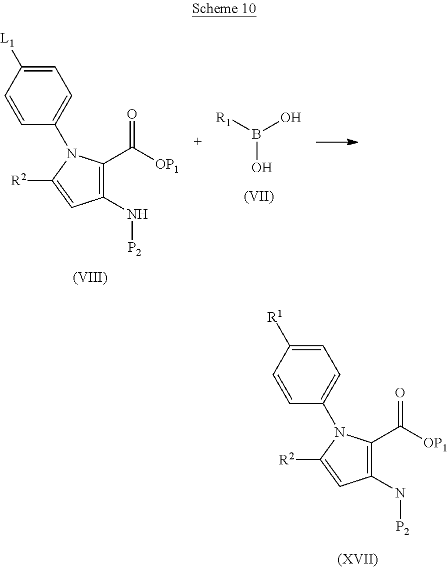

Compounds of formula (Ia) ((I) wherein R³ is —C₁₋₄alkylene(O)—O—C₁₋₄alkyl) or salts thereof, may be prepared according to reaction scheme 11 by reacting compounds of formula (Ib) ((I) wherein R³ is C₁ alkyl substituted by a —COOH group) or salts thereof, with the appropriate alcohol (C₁₋₄alkyl-OH) in the presence of hydrochloric acid (suitably at 80-90° C.).

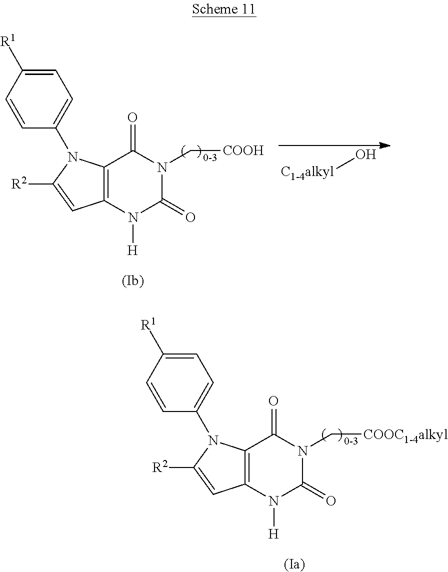

Compounds of formula (I) or salts thereof wherein R¹ and/or R³ is C₆₋₁₀aryl substituted by C₁₋₄alkylene(O)—O—C₁₋₄alkyl can be made from the corresponding carboxylic acid according to a similar method to the method of Scheme 11.

Compounds of formula (Ic), ((I), R³ is —C₁₋₄alkylene(O)NHC₁₋₄alkyl) or salts thereof, may be prepared according to reaction scheme 12 by reacting compounds of formula (Ib) or salts thereof, with the appropriate amine (C₁₋₄alkyl-NH) in the presence of EDCI or HATU in dichloromethane or a mixture of DCM and THFat RT.

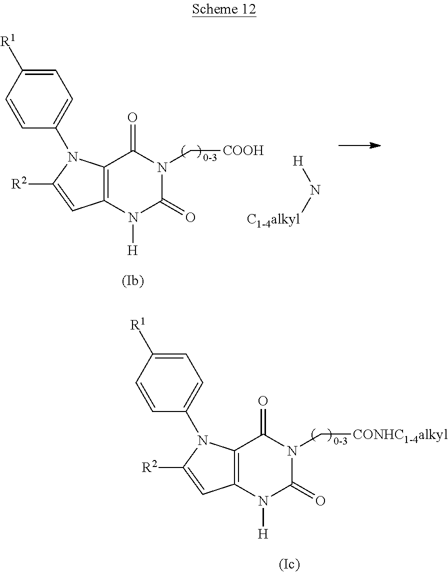

Compounds of formula (I) or salts thereof wherein R¹ and/or R³ is C₆₋₁₀aryl substituted by C₁₋₄alkylene(O)NR⁴C₁₋₄alkyl can be made from the corresponding carboxylic acid according to a similar method to the method of Scheme 12.

Compounds of formula (IIa) ((II) wherein R³ is H) or salts thereof may be prepared according to Scheme 13 by reacting compounds of formula (III) or salts thereof, wherein P₁ is a suitable protecting group such as alkyl (e.g. ethyl), with sodium cyanate in a mixture of acetic acid and water as solvents at 100° C.

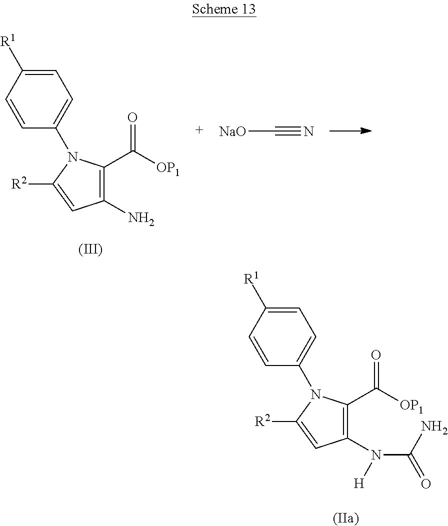

Further details for the preparation of compounds of formula (I) are found in the Examples section hereinafter.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Those skilled in the art will appreciate that in the preparation of compounds of formula (I) and/or solvates thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl).

The synthesis of the target compound is completed by removing any protecting groups, which are present in the penultimate intermediate using standard techniques, which are well-known to those skilled in the art. The final product is then purified, as necessary, using standard techniques such as silica gel chromatography, HPLC on silica gel, and the like or by recrystallization.

Various intermediate compounds used in the above-mentioned process, including but not limited to certain compounds of formulae (II), (XIV) and (XIVI) constitute a further aspect of the present invention.

### DEFINITIONS

AcOH acetic acid
BBr₃ boron tribromide
CH₃CN acetonitrile
CDCl₃ deuterated chloroform
DCM dichloromethane
DMF N,N-dimethylformamide
DMSO d6 deuterated dimethylsulfoxide
DMSO dimethylsulfoxide
Et₃N triethylamine
Et₂O diethyl ether
EtOAc/AcOEt ethyl acetate
EtOH ethanol
h hours
H₂SO₄ sulphuric acid
H₂O water
HATU O-(7-azabenzotriazol-1-yl)-1,1,3,3-tetramethyluronium hexafluorophosphate
HCl hydrochloric acid
HRMS high resolution mass spectroscopy
LCMS liquid chromatography mass spectroscopy
MeOH methanol
NaHCO₃ sodium hydrogen carbonate
NaOH sodium hydroxide
Na₂SO₄ sodium sulphate
NMR Nuclear magnetic resonance
RT room temperature
Rt retention time
Sat. saturated
SM starting material
THF tetrahydrofuran
TLC thin-layer chromatography

The compounds and processes of the present invention will be better understood in connection with the following examples, which are intended as an illustration only and not limiting the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Regardless of how the preparation of compounds are represented in the present specification no inference can be drawn that particular batches (or mixtures of two or more batches) of intermediates were used in the next stage of the preparation. The examples and intermediates are intended to illustrate the synthetic routes suitable for preparation of the same, to assist the skilled persons understanding of the present invention.

Where reference is made to the use of a “similar” or “analogous” procedure, as will be appreciated by those skilled in the art, such a procedure may involve minor variation, for example reaction temperature, reagent/solvent amount, reaction time, work-up conditions or chromatographic purification conditions.

### Analytical Method LC-MS

(a) Analytical HPLC was conducted on a X-terra MS C18 column (2.5 μm 3*30 mm id) eluting with 0.01M ammonium acetate in water (solvent A) and 100% acetonitrile (solvent B) using the following elution gradient: 0 to 4 minutes, 5% B to 100% B; 4 to 5 minutes, 100% B at a flow rate of 1.1 mL/min with a temperature of 40° C. The mass spectra (MS) were recorded on a Micromass ZQ-LC mass spectrometer using electrospray positive ionisation [ES+ve to give MH⁺ molecular ion] or electrospray negative ionisation [ES−ve to give (M−H)⁻ molecular ion] modes.

### OR

(b) Analytical HPLC was conducted on a X-Terra MS C18 column (3.5 μm 30×4.6 mm id) eluting with 0.01M ammonium acetate in water (solvent A) and 100% methanol (solvent B), using the following elution gradient 0 to 7.5 minutes 10 to 100% B, 7.5-10 minutes 100% B, 10.5-12 min 10% B at a flow rate of 1.4 ml/minute. The mass spectra (MS) were recorded on a Waters ZQ mass spectrometer using electrospray positive ionisation [ES− to give MH+ molecular ions] or electrospray negative ionisation [ES− to give (M−H)− molecular ions] modes.

### Analytical LC-HRMS

### Methods:

(a) Analytical HPLC was conducted on a LUNA 3u C18 column (2.5 μm 30×3 mm id) eluting with 0.01M ammonium acetate in water (solvent A) and 100% acetonitrile (solvent B) using the following elution gradient: 0 to 0.5 minutes, 5% B; 0.5 to 3.5 minutes, 5% B to 100% B; 3.5 to 4 minutes, 100% B; 4 to 4.5 minutes, 100% B to 5% B; 4.5 to 5.5 minutes, 5% B at a flowrate of 1.3 mL/min with a temperature of 40° C. The mass spectra (MS) were recorded on a Micromass LCT mass spectrometer using electrospray positive ionisation [ES+ve to give MH⁺ molecular ion] or electrospray negative ionisation [ES−ve to give (M−H)⁻ molecular ion] modes.

### OR

(b) Analytical HPLC was conducted on a X-Bridge C18 column (2.5 μm 30×3 mm id) eluting with 0.01M ammonium acetate in water (solvent A) and 100% acetonitrile (solvent B) using the following elution gradient: 0 to 0.5 minutes, 5% B; 0.5 to 3.5 minutes, 5% B to 100% B; 3.5 to 4 minutes, 100% B; 4 to 4.5 minutes, 100% B to 5% B; 4.5 to 5.5 minutes, 5% B at a flowrate of 1.3 mL/min with a temperature of 40° C. The mass spectra (MS) were recorded on a Micromass LCT mass spectrometer using electrospray positive ionisation [ES+ve to give MH⁺ molecular ion] or electrospray negative ionisation [ES−ve to give (M−H)⁻ molecular ion] modes.

¹H NMR spectra were acquired on a 300 MHz and 400 MHz Bruker spectrometer. Sample was dissolved in DMSO-d6 or CDCl₃ and chemical shifts were reported in ppm relative to the TMS signal at δ=0 ppm. Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

The following non-limiting Examples illustrate the present invention.

### Intermediate 1

### Ethyl 3-(acetylamino)-1H-pyrrole-2-carboxylate

To a suspension of ethyl 3-amino-1H-pyrrole-2-carboxylate hydrochloride (1 g, 4.98 mmol, commercially available from Combi-Blocks) in DCM (50 mL) at 0° C. was added drop-wise triethylamine (2 mL, 14.43 mmol) and acetyl chloride (0.45 mL, 6.31 mmol). The reaction mixture was then stirred from 0° C. to RT for 12 hours before being quenched with 1N HCl. The organic layer was separated and washed successively with sat. NaHCO₃ and brine, dried over Na₂SO₄, filtered and concentrated under reduced pressure. The product was purified by chromatography on an Isco Companion RF. The sample was loaded on 100 g Biotage silica column and then the purification was carried out using DCM/MeOH 100/0 to 90/10. The appropriate fractions were combined and evaporated in vacuo to give the required product ethyl 3-(acetylamino)-1H-pyrrole-2-carboxylate (0.99 g, 5.05 mmol, 100% yield) as a yellow solid.

LCMS: (M+H)⁺=197; Rt=1.93 min.

### Intermediate 2

### Ethyl 3-(acetylamino)-1-(4-bromophenyl)-1H-pyrrole-2-carboxylate

Copper (II) acetate (1.37 g, 7.57 mmol) was added to a solution of 4-bromophenylboronic acid (2.03 g, 10.09 mmol), ethyl 3-(acetylamino)-1H-pyrrole-2-carboxylate (0.99 g, 5.05 mmol; Intermediate 1) and pyridine (0.81 mL, 10.04 mmol) in DCM (20 mL) at RT. The reaction mixture was stirred for 24 hours. 4-bromophenylboronic acid (2.03 g, 10.09 mmol), copper (II) acetate (1.38 g, 7.57 mmol) and pyridine (0.81 mL, 10.04 mmol) were added again in the same order and the mixture was stirred for another 72 hours. The reaction mixture was then concentrated under reduced pressure. The crude extract was then purified by chromatography on an Isco Companion RF. The sample was loaded on 100 g Biotage silica column then the purification was carried out using cyclohexane/EtOAc 100/0 to 50/50. The appropriate fractions were combined and concentrated in vacuo to give the required product ethyl 3-(acetylamino)-1-(4-bromophenyl)-1H-pyrrole-2-carboxylate (1.36 g, 3.87 mmol, 77% yield) as a colorless oil which solidified.

LCMS: (M+H)⁺=351, 353; Rt=3.28 min.

### Intermediate 3

### Ethyl 3-amino-1-(4-bromophenyl)-1H-pyrrole-2-carboxylate hydrochloride

A solution of ethyl 3-(acetylamino)-1-(4-bromophenyl)-1H-pyrrole-2-carboxylate (Intermediate 2; 1.15 g, 3.27 mmol) and concentrated HCl (4 mL, 48.7 mmol) in ethanol (50 mL) was refluxed for 2 hours before being concentrated under reduced pressure. The crude solid was triturated in hot CH₃CN and the solid filtered and dried to give the desired compound ethyl 3-amino-1-(4-bromophenyl)-1H-pyrrole-2-carboxylate hydrochloride (0.62 g, 1.79 mmol, 54.8% yield) as a white solid.

LCMS: (M+H)⁺=309, 311; Rt=3.19 min.

### Intermediate 4

### Ethyl 3-amino-1-[2′-hydroxy-3′-(methyloxy)-4-biphenylyl]-1H-pyrrole-2-carboxylate hydrochloride

To a suspension of ethyl 3-amino-1-(4-bromophenyl)-1H-pyrrole-2-carboxylate hydrochloride (3.5 g, 11.32 mmol; Intermediate 3), in 1,4-dioxane (150 mL), water (150 mL) were added [2-hydroxy-3-(methyloxy)phenyl]boronic acid (11.41 g, 67.9 mmol), palladium tetrakis (0.13 g, 0.11 mmol), cesium carbonate (12.91 g, 39.6 mmol). The reaction was stirred at reflux for 2 hours. After cooling, the mixture was filtered and treated with a 1N HCl solution. The dioxane was evaporated and the compound was precipitated from the acid phase. The solid was filtered, washed with water and dried. The title compound ethyl 3-amino-1-[2′-hydroxy-3′-(methyloxy)-4-biphenylyl]-1H-pyrrole-2-carboxylate hydrochloride was obtained (3.9 g, 11.07 mmol, 98% yield) as a cream solid.

LCMS: (M+H)⁺=353; Rt=3.25 min.

### Intermediate 5

### Ethyl 3-(acetylamino)-5-chloro-1H-pyrrole-2-carboxylate

Ethyl 3-(acetylamino)-1H-pyrrole-2-carboxylate (intermediate 1; 40 g, 204 mmol) was dissolved in chloroform (250 mL) and N-chlorosuccinimide (28.6 g, 214 mmol) was added portion-wise. The mixture was stirred at RT for 1 hour, and was warmed to 35° C. during 2 hours. The mixture was then poured into water and extracted with DCM, dried over sodium sulfate, and concentrated in vacuo. The mixture was triturated in DCM and the precipitate was filtered, washed with a small of DCM and washed with Et₂O to give ethyl 3-(acetylamino)-5-chloro-1H-pyrrole-2-carboxylate (20 g, 42% yield) as a white solid.

LCMS: (M+H)⁺=231; Rt=2.32 min.

### Intermediate 6

### Ethyl 3-(acetylamino)-1-(4-bromophenyl)-5-chloro-1H-pyrrole-2-carboxylate

To a suspension of ethyl 3-(acetylamino)-5-chloro-1H-pyrrole-2-carboxylate (Intermediate 5; 200 mg, 0.87 mmol) and molecular sieves 4 Å (500 mg, 0.87 mmol) in DCM (5 mL) was added (4-bromophenyl)boronic acid (192 mg, 0.95 mmol), copper (II) acetate (173 mg, 0.95 mmol) and Et₃N (0.18 mL, 1.30 mmol). The reaction mixture was stirred at room temperature overnight. (4-Bromophenyl)boronic acid (192 mg, 0.95 mmol, 4 equiv. in total) was added every 2 hours and the reaction was complete. The mixture was filtered on silica pad (DCM and MeOH) and the filtrate was concentrated. The residue was purified by chromatography on silica gel (interchim 12 g) (DCM/MeOH 100/0 to 99/1) to give the product ethyl 3-(acetylamino)-1-(4-bromophenyl)-5-chloro-1H-pyrrole-2-carboxylate (350 mg, 0.86 mmol, 99% yield) as a light yellow oil.

LCMS: (M+H)⁺=385, 387; Rt=3.83 min.

### Intermediate 7

### Ethyl 3-amino-1-(4-bromophenyl)-5-chloro-1H-pyrrole-2-carboxylate hydrochloride

To a solution of ethyl 3-(acetylamino)-1-(4-bromophenyl)-5-chloro-1H-pyrrole-2-carboxylate (Intermediate 6; 29.3 g, 76 mmol) in ethanol (630 mL) was added concentrated HCl (31 mL, 0.38 mol). The mixture was refluxed for 4 hours before being concentrated in vacuo. The crude product was triturated in diethyl ether to give ethyl 3-amino-1-(4-bromophenyl)-5-chloro-1H-pyrrole-2-carboxylate hydrochloride (27.09 g, 93.8% yield) as a grey solid.

¹H NMR (DMSO d⁶, 400 MHz): δ 7.67 (d, 2H), 7.23 (d, 2H), 6.05 (brs, 1H), 3.95 (q, 2H), 0.97 (t, 3H).

### Intermediate 8

### Ethyl 3-amino-5-chloro-1-[2′-hydroxy-3′-(methyloxy)-4-biphenylyl]-1H-pyrrole-2-carboxylate

To a solution of ethyl 3-amino-1-(4-bromophenyl)-5-chloro-1H-pyrrole-2-carboxylate hydrochloride (Intermediate 7; 14.85 g, 39.1 mmol) in 1.4-dioxane (412 mL) and water (50 mL) [2-hydroxy-3-(methyloxy)phenyl]boronic acid (9.87 g, 58.7 mmol), cesium carbonate (38.21 g, 117 mmol) under argon, was added palladium tetrakis (451 mg, 391 μmol). The reaction was heated at reflux for one hour. The reaction mixture was concentrated in vacuo, dissolved in DCM and washed with water. The organic layer was dried over anhydrous sodium sulfate, filtered and concentrated in vacuo. The residue was purified by chromatography on silica gel (petroleum ether/EtOAc 90/10, 80/20 and 70/30) to give the title compound, ethyl 3-amino-5-chloro-1-[2′-hydroxy-3′-(methyloxy)-4-biphenylyl]-1H-pyrrole-2-carboxylate, (13.64 g, 90.3% yield) as a beige solid.

¹H NMR (DMSO d⁶, 400 MHz): δ 8.76 (s, 1H), 7.6 (d, 2H), 7.2 (d, 2H), 6.98 (d, 1H), 6.9 (m, 2H), 5.88 (s, 1H), 5.6 (brs, 2H), 3.9 (q, 2H), 3.86 (s, 3H), 0.91 (t, 3H).

Intermediates 9 to 11 of the general formula below were prepared by methods analogous to that described for intermediate 8 using the appropriate boronic acid or dioxoborolane compound.

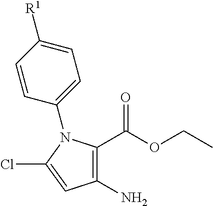

TABLE 1

| | | | | Physical |
|---|---|---|---|---|
| Intermediate | Name | Starting material | R¹ | data |
| Intermediate 9 | Ethyl 3-amino- 5-chloro-1-(2′- hydroxy-3′- methyl-4- biphenylyl)-1H- pyrrole-2- carboxylate | Ethyl 3-amino-1-(4- bromophenyl)-5-chloro- 1H-pyrrole-2-carboxylate hydrochloride (Intermediate 7) and (2- hydroxy-3- methylphenyl)boronic acid | 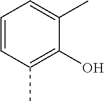 | LCMS: (M + H)⁺ = 371; Rt = 3.62 min. |
| Intermediate 10 | Ethyl 3-amino- 1-[2′,3′- bis(methyloxy)- 4-biphenylyl]- 5-chloro-1H- pyrrole-2- carboxylate | Ethyl 3-amino-1-(4- bromophenyl)-5-chloro- 1H-pyrrole-2-carboxylate hydrochloride (Intermediate 7) [2,3- bis(methyloxy)phenyl] boronic acid | 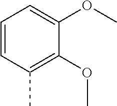 | LCMS: (M + H)⁺ = 401; Rt = 3.71 min. |
| Intermediate 11 | Ethyl 3-amino- 5-chloro-1-(2′- hydroxy-4- biphenylyl)-1H- pyrrole-2- carboxylate | Ethyl 3-amino-1-(4- bromophenyl-5-chloro- 1H-pyrrole-2-carboxylate hydrochloride (Intermediate 7) and 2- (4,4,5,5-tetramethyl-1,3,2- dioxaborolan-2-yl)phenol | 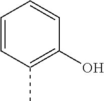 | LCMS: (M + H)⁺ = 357; Rt = 3.30 min. |

### Intermediate 12

### Ethyl 5-chloro-3-[({[3-(ethyloxy)-3-oxopropyl]amino}carbonyl)amino]-1-[2′-hydroxy-3′-(methyloxy)-4-biphenylyl]-1H-pyrrole-2-carboxylate

To a solution of ethyl 3-amino-5-chloro-1-[2′-hydroxy-3′-(methyloxy)-4-biphenylyl]-1H-pyrrole-2-carboxylate (Intermediate 8; 0.45 g, 1.16 mmol) in toluene (30 mL) was added ethyl N-(oxomethylidene)-β-alaninate (0.25 g, 1.75 mmol) and the reaction was stirred at 80° C. overnight. After cooling, the solvent was concentrated in vacuo and the compound was purified by flash chromatography using cyclohexane/EtOAc as gradient to give the title compound, ethyl 5-chloro-3-[({[3-(ethyloxy)-3-oxopropyl]amino}carbonyl)amino]-1-[2′-hydroxy-3′-(methyloxy)-4-biphenylyl]-1H-pyrrole-2-carboxylate (430 mg, 0.81 mmol, 69.7% yield) as a colorless foam.

LCMS: (M−H)⁺=528; Rt=3.68 min

Intermediates 13 to 15 of the general formula below were prepared by methods analogous to that described for intermediate 12 using the appropriate isocyanate.

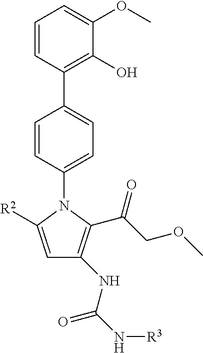

TABLE 2

| | | | | | Physical |
|---|---|---|---|---|---|
| Intermediate | Name | Starting material | R² | R³ | data |
| Intermediate 13 | Ethyl 5- chloro-3-[({[4- (ethyloxy)-4- oxobutyl]amino} carbonyl) amino]-1-[2′- hydroxy-3′- (methyloxy) 4-biphenylyl]- 1H-pyrrole-2- carboxylate | Ethyl 3-amino-5- chloro-1-[2′- hydroxy-3′- (methyloxy)-4- biphenylyl]-1H- pyrrole-2- carboxylate, (Intermediate 8), and ethyl-4 isocyanatobutanoate | Cl | 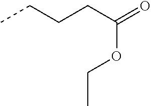 | LCMS: (M − H)⁺ = 542; Rt = 3.69 min. |
| Intermediate 14 | Ethyl 5- chloro-3-[({[2- (ethyloxy)-2- oxoethyl]amino} carbonyl) amino]-1-[2′- hydroxy-3′- (methyloxy)- 4-biphenylyl]- 1H-pyrrole-2- carboxylate | Ethyl 3-amino-5- chloro-1-[2′- hydroxy-3′- (methyloxy)-4- biphenylyl]-1H- pyrrole-2- carboxylate, (Intermediate 8), and ethyl N- (oxomethylidene) glycinate | Cl | 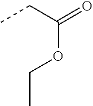 | LCMS: (M + H)⁺ = 516; Rt = 3.61 min. |
| Intermediate 15 | Ethyl 3-[({[3- (ethyloxy)-3- oxopropyl]amino} carbonyl) amino]-1-[2′- hydroxy-3′- (methyloxy)- 4-biphenylyl]- 1H-pyrrole-2- carboxylate | Ethyl 3-amino-1-[2′- hydroxy-3′- (methyloxy)-4- biphenylyl]-1H- pyrrole-2- carboxylate hydrochloride (Intermediate 4), and ethyl N- (oxomethylidene)- β-alaninate | H | 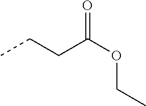 | LCMS: (M + H)⁺ = 496; Rt = 3.41 min. |

The intermediate 16 was prepared by methods analogous to that described for intermediate 12 using the appropriate isocyanate.

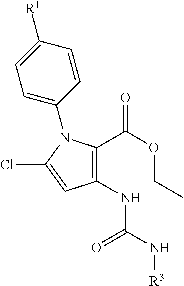

TABLE 3

| | | Starting | | | Physical |
|---|---|---|---|---|---|
| Intermediate | Name | material | R¹ | R³ | data |
| Intermediate 16 | Ethyl 5- chloro-3- [({[3- (ethyloxy)-3- oxopropyl] amino}carbonyl) amino]-1-(2′- hydroxy-3′- methyl-4- biphenylyl)- 1H-pyrrole-2- carboxylate | Ethyl 3-amino- 5-chloro-1-(2′- hydroxy-3′- methyl-4- biphenylyl)-1H- pyrrole-2- carboxylate (Intermediate 9) and ethyl N- (oxomethylidene)- β-alaninate | 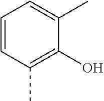 | 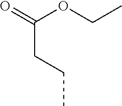 | LCMS: (M + H)⁺ = 514, Rt = 3.74 min. |

### Intermediate 17

### Ethyl 3-({[(2-fluorophenyl)amino]carbonyl}amino)-1-[2′-hydroxy-3′-(methyloxy)-4-biphenylyl]-1H-pyrrole-2-carboxylate

To a solution of ethyl 3-amino-1-[2′-hydroxy-3′-(methyloxy)-4-biphenylyl]-1H-pyrrole-2-carboxylate hydrochloride (Intermediate 4; 150 mg, 0.43 mmol) in toluene (10 mL) was added 1-fluoro-2-isocyanatobenzene (0.06 mL, 0.51 mmol) and the reaction was stirred at reflux for 48 hours. After cooling, the solvent was evaporated in vacuo and the residue was purified by flash chromatography using DCM/MeOH 95/5 as gradient just to remove the polar impurities. The title compound ethyl 3-({[(2-fluorophenyl)amino]carbonyl}amino)-1-[2′-hydroxy-3′-(methyloxy)-4-biphenylyl]-1H-pyrrole-2-carboxylate, (170 mg, 0.35 mmol, 82% yield) was obtained.

LCMS: (M−H)⁺=488; Rt=3.81 min.

Intermediates 18 and 19 were prepared by methods analogous to that described for Intermediate 17 using the appropriate isocyanate.

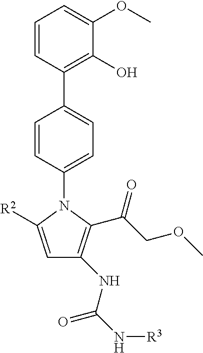

TABLE 4

| | | | | | Physical |
|---|---|---|---|---|---|
| Intermediate | Name | Starting material | R² | R³ | data |
| Intermediate 18 | Ethyl 3- ({[(2,3- dimethylphen yl)amino] carbonyl} amino)- 1-[2′- hydroxy-3′- (methyloxy)- 4-biphenylyl]- 1H-pyrrole-2- carboxylate | Ethyl 3-amino-1- [2′-hydroxy-3′- (methyloxy)-4- biphenylyl]-1H- pyrrole-2- carboxylate hydrochloride (Intermediate 4), and 1-isocyanato- 2,3- dimethylbenzene | H | 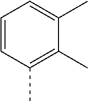 | LCMS: (M + H)⁺ = 500; Rt = 3.78 min. |
| Intermediate 19 | Ethyl 3-({[(3- chloro-2- methylphenyl) amino] carbonyl} amino)-1- [2′-hydroxy- 3′- (methyloxy)- 4-biphenylyl]- 1H-pyrrole-2- carboxylate | Ethyl 3-amino-1- [2′-hydroxy-3′- (methyloxy)-4- biphenylyl]-1H- pyrrole-2- carboxylate hydrochloride (Intermediate 4), and 1-chloro-3- isocyanato-2- methylbenzene | H | 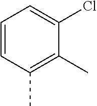 | LCMS: (M + H)⁺ = 520; Rt = 3.92 min. |

### Intermediate 20

### Ethyl 5-chloro-1-[2′-hydroxy-3′-(methyloxy)-4-biphenylyl]-3-{[(Phenylamino)carbonyl]amino}-1H-pyrrole-2-carboxylate

To a solution of ethyl 3-amino-5-chloro-1-[2′-hydroxy-3′-(methyloxy)-4-biphenylyl]-1H-pyrrole-2-carboxylate (Intermediate 8; 100 mg, 0.26 mmol) in xylene (5 mL) was added isocyanatobenzene (0.04 mL, 0.34 mmol). The reaction was performed under microwave irradiation at 140° C. for 15 min. The suspension was filtered to give the title compound ethyl 5-chloro-1-[2′-hydroxy-3′-(methyloxy)-4-biphenylyl]-3-{[(phenylamino)carbonyl]amino}-1H-pyrrole-2-carboxylate (78 mg, 0.15 mmol, 59.6% yield) as an off-white solid.

LCMS: (M+H)⁺=506; Rt=4.02 min

Intermediates 21 to 35 were prepared by methods analogous to that described for intermediate 20 using the appropriate isocyanate.

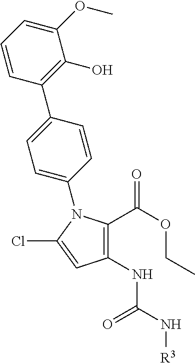

TABLE 5

| | | | | Physical |
|---|---|---|---|---|
| Intermediate | Name | Starting material | R³ | data |
| Intermediate 21 | Ethyl 5-chloro-1- [2′-hydroxy-3′- (methyloxy)-4- biphenylyl]-3-({[(2- methylphenyl) amino]carbonyl} amino)- 1H-pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 1-isocyanato-2- methylbenzene | 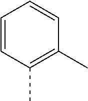 | LCMS: (M + H)⁺ = no mass detected, Rt = 3.97 min. |
| Intermediate 22 | Ethyl 5-chloro-3- ({[(2- chlorophenyl)amino] carbonyl}amino)- 1-[2′-hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8), and 1-chloro-2- isocyanatobenzene | 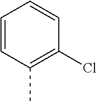 | LCMS: (M + H)⁺ = 540 542; Rt = 4.12 min |
| Intermediate 23 | Ethyl 5-chloro-3- ({[(3- chlorophenyl)amino] carbonyl}amino)- 1-[2′-hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 1-chloro-3- isocyanatobenzene | 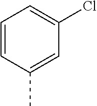 | LCMS: (M + H)⁺ = 540 542; Rt = 4.28 min |
| Intermediate 24 | Ethyl 5-chloro-3- ({[(2- fluorophenyl)amino] carbonyl}amino)- 1-[2′-hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 1-fluoro-2- isocyanatobenzene | 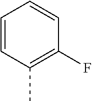 | LCMS: (M + H)⁺ = 524; Rt = 4.04 min |
| Intermediate 25 | Ethyl 5-chloro-1- [2′-hydroxy-3′- (methyloxy)-4- biphenylyl]-3-[({[3- (methyloxy)phenyl] amino}carbonyl) amino]-1H-pyrrole- 2-carboxylate | Ethyl 3-amino-5- chloro-1-[2′-hydroxy- 3′-(methyloxy)-4- biphenylyl]-1H- pyrrole-2-carboxylate (Intermediate 8) and 1-isocyanato-3- (methyloxy)benzene | 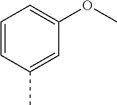 | LCMS: (M + H)⁺ = 536; Rt = 4.00 min |
| Intermediate 26 | Ethyl 5-chloro-1- [2′-hydroxy-3′- (methyloxy)-4- biphenylyl]-3-[({[4- (methyloxy)phenyl] amino}carbonyl) amino]-1H-pyrrole- 2-carboxylate | Ethyl 3-amino-5- chloro-1-[2′-hydroxy- 3′-(methyloxy)-4- biphenylyl]-1H- pyrrole-2-carboxylate (Intermediate 8) and 1-isocyanato-4- (methyloxy)benzene | 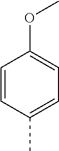 | LCMS: (M + H)⁺ = 536; Rt = 3.91 min |
| Intermediate 27 | Ethyl 5-chloro-3- ({[(4- fluorophenyl)amino] carbonyl}amino)- 1-[2′-hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 1-fluoro-4- isocyanatobenzene | 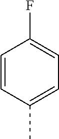 | LCMS: (M + H)⁺ = 524; Rt = 4.05 min |
| Intermediate 28 | Ethyl 5-chloro-3- ({[(4- chlorophenyl)amino] carbonyl}amino)- 1-[2′-hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 1-chloro-4- isocyanatobenzene | 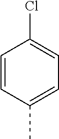 | LCMS: (M + H)⁺ = 540 542; Rt = 4.25 min |
| Intermediate 29 | Ethyl 5-chloro-1- [2′-hydroxy-3′- (methyloxy)-4- biphenylyl]-3-({[(4- methylphenyl)amino] carbonyl}amino)- 1H-pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 1-isocyanato-4- methylbenzene | 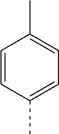 | LCMS: (M + H)⁺ = 520; Rt = 4.12 min |
| Intermediate 30 | Ethyl 5-chloro-1- [2′-hydroxy-3′- (methyloxy)-4- biphenylyl]-3-({[(3- methylphenyl)amino] carbonyl}amino)- 1H-pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 1-isocyanato-3- methylbenzene | 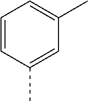 | LCMS: (M + H)⁺ = 520; Rt = 4.12 min |
| Intermediate 31 | Ethyl 5-chloro-3- ({[(4- cyanophenyl)amino] carbonyl}amino)- 1-[2′-hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 4- isocyanatobenzonitrile | 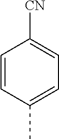 | LCMS: (M + H)⁺ = 531; Rt = 3.96 min |
| Intermediate 32 | Ethyl 5-chloro-1- [2′-hydroxy-3′- (methyloxy)-4- biphenylyl]-3-({[(2- methyl-6- nitrophenyl)amino] carbonyl}amino)- 1H-pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 2-methyl-6- nitrophenylisocyanate | 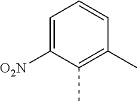 | LCMS: (M + H)⁺ = 565; Rt = 3.86 min |
| Intermediate 33 | Ethyl 5-chloro-3- [({[2-fluoro-3- (trifluoromethyl) phenyl]amino} carbonyl)amino]- 1-[2′- hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 2-fluoro-1- isocyanato-3- (trifluoromethyl) benzene | 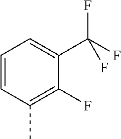 | LCMS: (M + H)⁺ = 592; Rt = 4.30 min |
| Intermediate 34 | Ethyl 5-chloro-3- ({[(2,6- dimethylphenyl) amino]carbonyl} amino)-1-[2′- hydroxy- 3′-(methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 2-isocyanato-1,3- dimethylbenzene | 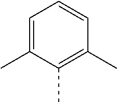 | LCMS: (M + H)⁺ = 534; Rt = 3.92 min |
| Intermediate 35 | Ethyl 5-chloro-1- [2′-hydroxy-3′- (methyloxy)-4- biphenylyl]-3-({[(2- methyl-3- nitrophenyl)amino] carbonyl}amino)- 1H-pyrrole-2- carboxylate | Ethyl 3-amino-5- chloro-1-[2′-hydroxy- 3′-(methyloxy)-4- biphenylyl]-1H- pyrrole-2-carboxylate (Intermediate 8) and 2-methyl-3- nitrophenylisocyanate | 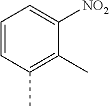 | LCMS: (M + H)⁺ = 565; Rt = 3.99 min |

Intermediate 36 was prepared by methods analogous to that described for intermediate 20 using the appropriate intermediates and isocyanate.

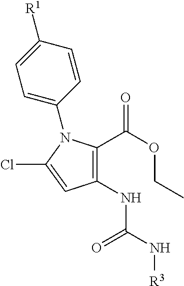

TABLE 6

| | | Starting | | | Physical |
|---|---|---|---|---|---|
| Intermediate | Name | material | R¹ | R³ | data |
| Intermediate 36 | Ethyl 5- chloro-3- [({[3- (ethyloxy)-3- oxopropyl] amino} carbonyl) amino]-1- (2′-hydroxy- 4- biphenylyl)- 1H-pyrrole- 2- carboxylate | Ethyl 3-amino- 5-chloro-1-(2′- hydroxy-4- biphenylyl)-1H- pyrrole-2- carboxylate (Intermediate 11), and ethyl N- (oxomethylidene)- β-alaninate | 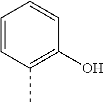 | 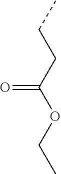 | LCMS: (M + H)⁺ = 500, Rt = 3.44 min. |

The intermediates 37 to 56 were prepared by methods analogous to that described for intermediate 20 using the appropriate isocyanate in toluene at 80° C.

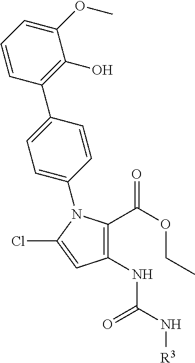

TABLE 7

| | | | | Physical |
|---|---|---|---|---|
| Intermediate | Name | Starting material | R³ | data |
| Intermediate 37 | Ethyl 5-chloro- 3-({[(2,6- dichlorophenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1,3-dichloro-2- isocyanatobenzene | 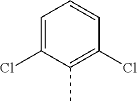 | LCMS: (M + H)⁺ = 576; Rt = 3.92 min |
| Intermediate 38 | Ethyl 5-chloro- 3-({[(3-chloro-2- fluorophenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-chloro-2-fluoro-3- isocyanatobenzene | 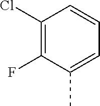 | LCMS: (M + H)⁺ = 558; Rt = 4.23 min |
| Intermediate 39 | Ethyl 5-chloro- 3-({[(3-fluoro-2- methylphenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-fluoro-3- isocyanato-2- methylbenzene | 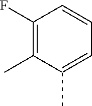 | LCMS: (M + H)⁺ = 538; Rt = 4.02 min |
| Intermediate 40 | Ethyl 5-chloro- 3-({[(2,3- dimethylphenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-isocyanato-2,3- dimethylbenzene | 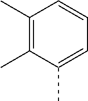 | LCMS: (M + H)⁺ = 534; Rt = 4.00 min |
| Intermediate 41 | Ethyl 5-chloro- 3-({[(3- cyanophenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 3- isocyanatobenzonitrile | 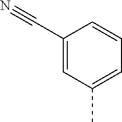 | LCMS: (M + H)⁺ = 531; Rt = 4.01 min |
| Intermediate 42 | Ethyl 5-chloro- 3-({[(3-chloro-2- methylphenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-chloro-3- isocyanato-2- methylbenzene | 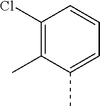 | LCMS: (M + H)⁺ = 554; Rt = 4.14 min |
| Intermediate 43 | Ethyl 5-chloro- 3-({[(2-chloro-6- methylphenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-chloro-2- isocyanato-3- methylbenzene | 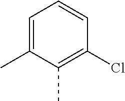 | LCMS: (M + H)⁺ = 554; Rt = 3.95 min |
| Intermediate 44 | Ethyl 5-chloro- 3-({[(2,6- difluorophenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1,3-difluoro-2- isocyanatobenzene | 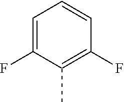 | LCMS: (M + H)⁺ = 542; Rt = 3.83 min |
| Intermediate 45 | Ethyl 5-chloro- 1-[2′-hydroxy- 3′-(methyloxy)- 4-biphenylyl]-3- [({[3- (trifluoromethyl) phenyl]amino} carbonyl)amino]- 1H-pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-isocyanato-3- (trifluoromethyl) benzene | 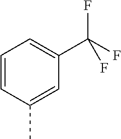 | LCMS: (M + H)⁺ = 574; Rt = 4.29 min |
| Intermediate 46 | Ethyl 5-chloro- 1-[2′-hydroxy- 3′-(methyloxy)- 4-biphenylyl]-3- {[({2-[(2- methylacryloyl) oxy]ethyl}amino) carbonyl]amino}- 1H-pyrrole- 2-carboxylate | Ethyl 3-amino-5- chloro-1-[2′- hydroxy-3′- (methyloxy)-4- biphenylyl]-1H- pyrrole-2- carboxylate (Intermediate 8) and 2-isocyanatoethyl 2- methyl-2- propenoate | 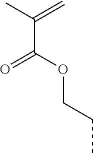 | ¹H NMR: (CDCl₃, 300 MHz) δ 8.87 (s, 1H), 7.61 (d, 2H), 7.16 (d, 2H), 6.90 (m, 3H), 6.07 (s, 1H), 5.88 (s, 1H), 5.53 (brs, 1H), 4.22 (t, 2H), 3.89 (m, 5H), 3.5 (m, 2H), 1.89 (s, 3H), 0.76 (t, 3H). |
| Intermediate 47 | Ethyl 5-chloro- 1-[2′-hydroxy- 3′-(methyloxy)- 4-biphenylyl]-3- [({[2- (methyloxy)ethyl] amino}carbonyl) amino]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-isocyanato-2- (methyloxy)ethane | 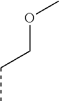 | LCMS: (M + H)⁺ = 488; Rt = 3.45 min |
| Intermediate 48 | Ethyl 5-chloro- 3-({[(2,3- dichlorophenyl) amino]carbonyl} amino)-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1,2-dichloro-3- isocyanatobenzene | 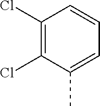 | LCMS: (M − H)⁺ = 572-574; Rt = 4.29 min |
| Intermediate 49 | Ethyl 5-chloro- 1-[2′-hydroxy- 3′-(methyloxy)- 4-biphenylyl]-3- [({[3- (methyloxy) propyl]amino} carbonyl)amino]- 1H-pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-isocyanato-3- (methyloxy)propane | 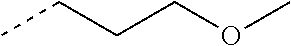 | LCMS: (M − H)⁺ = 500-502; Rt = 3.48 min |
| Intermediate 50 | Ethyl 3-[({[3,4- bis(methyloxy) phenyl]amino} carbonyl)amino]- 5-chloro-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 3,4- dimethoxyphenyl isocyanate | 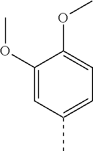 | LCMS: (M + H)⁺ = 566; Rt = 3.72 min |
| Intermediate 51 | Ethyl 3-[({[3,5- bis(methyloxy) phenyl]amino} carbonyl)amino]- 5-chloro-1-[2′- hydroxy-3′- (methyloxy)-4- biphenylyl]-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 3,5- dimethoxyphenyl isocyanate | 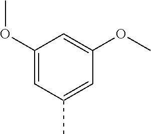 | LCMS: (M + H)⁺ = 566; Rt = 3.94 min |
| Intermediate 52 | Ethyl 5-chloro- 1-[2′-hydroxy- 3′-(methyloxy)- 4-biphenylyl]-3- {[({2-methyl-3- [(methyloxy) carbonyl]phenyl} amino)carbonyl] amino}-1H- pyrrole-2- carboxylate | Ethyl 3-amino-5- chloro-1-[2′- hydroxy-3′- (methyloxy)-4- biphenylyl]-1H- pyrrole-2- carboxylate (intermediate 8) and methyl-3- isocyanato-2- methylbenzoate | 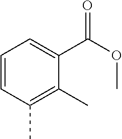 | LCMS: (M + H)⁺ = 578; Rt = 4.01 min |
| Intermediate 53 | Ethyl 5-chloro- 1-[2′-hydroxy- 3′-(methyloxy)- 4-biphenylyl]-3- {[(2- naphthalenylamino) carbonyl] amino}-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 2- isocyanatonaphthalene | 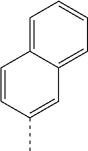 | LCMS: (M + H)⁺ = 556; Rt = 4.17 min |
| Intermediate 54 | Ethyl 5-chloro- 1-[2′-hydroxy- 3′-(methyloxy)- 4-biphenylyl]-3- [({[2- (methyloxy) phenyl]amino} carbonyl)amino]- 1H-pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-isocyanato-2- (methyloxy)benzene | 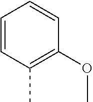 | LCMS: (M + H)⁺ = 536; Rt = 3.71 min |
| Intermediate 55 | Ethyl 5-chloro- 1-[2′-hydroxy- 3′-(methyloxy)- 4-biphenylyl]-3- [({[2-methyl-4- (methyloxy) phenyl]amino} carbonyl)amino]- 1H-pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1-isocyanato-2- methyl-4- (methyloxy)benzene | 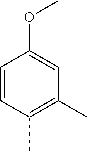 | LCMS: (M − H)⁺ = 548; Rt = 3.81 min |
| Intermediate 56 | Ethyl 5-chloro- 1-[2′-hydroxy- 3′-(methyloxy)- 4-biphenylyl]-3- {[(1- naphthalenylamino) carbonyl] amino}-1H- pyrrole-2- carboxylate | Ethyl 3-amino-5- chloro-1-[2′- hydroxy-3′- (methyloxy)-4- biphenylyl]-1H- pyrrole-2- carboxylate (Intermediate 8) and 1- isocyanatonaphthalene | 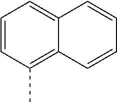 | LCMS: (M − H)⁺ = 554; Rt = 3.98 min |

### Intermediate 57

### 5-(4-Bromophenyl)-1H-pyrrolo[3,2-d]pyrimidine-2,4(3H,5H)-dione

A mixture of ethyl 3-amino-1-(4-bromophenyl)-1H-pyrrole-2-carboxylate hydrochloride (Intermediate 3; 500 mg, 1.62 mmol), and urea (1748 mg, 29.1 mmol) were heated to 250° C. for 30 min in a sand bath. After cooling, the product was washed with a mixture of ethanol/water and filtered to give the title compound 5-(4-bromophenyl)-1H-pyrrolo[3,2-d]pyrimidine-2,4(3H,5H)-dione (400 mg, 1.31 mmol, 81% yield) as a cream powder.

The crude product will be used in next step without further purification.

LCMS: (M+H)⁺=306-308; Rt=3.97 min.

### Intermediate 58

### Ethyl 3-[(aminocarbonyl)amino]-1-(4-bromophenyl)-5-chloro-1H-Pyrrole-2-carboxylate

To a solution of ethyl 3-amino-1-(4-bromophenyl)-5-chloro-1H-pyrrole-2-carboxylate hydrochloride (Intermediate 7; 200 mg, 0.53 mmol) in a mixture of acetic acid (10 mL)/water (10 mL), was added sodium cyanate (51.3 mg, 0.79 mmol). The reaction was stirred at 100° C. for 18 hours. The solvents were evaporated in vacuo and the residue was dissolved in diethyl ether, washed with a 1N HCl solution. The organic phase was concentrated in vacuo to give the title compound, ethyl 3-[(aminocarbonyl)amino]-1-(4-bromophenyl)-5-chloro-1H-pyrrole-2-carboxylate was obtained as a pale yellow oil, (150 mg, 0.39 mmol, 73.7% yield).

LCMS: (M+H)⁺=386-388; Rt=3.28 min.

### Intermediate 59

### 5-(4-Bromophenyl)-6-chloro-1H-pyrrolo[3,2-d]pyrimidine-2,4(3H,5H)-dione

To a solution of sodium (8.92 mg, 0.39 mmol) in ethanol (50 mL) was added ethyl 3-[(aminocarbonyl)amino]-1-(4-bromophenyl)-5-chloro-1H-pyrrole-2-carboxylate (Intermediate 58; 150 mg, 0.39 mmol). The reaction was stirred at 90° C. for 8 hours. The reaction mixture was concentrated in vacuo, taken in a 1N HCl solution until precipitation. The product was filtered, dried, to give the title compound 5-(4-bromophenyl)-6-chloro-1H-pyrrolo[3,2-d]pyrimidine-2,4(3H,5H)-dione (45 mg, 0.13 mmol, 34.1% yield) as a pale yellow solid.

LCMS: (M+H)⁺=340-342; Rt=2.67 min.

### Intermediate 60

### 5-[2′-Fluoro-6′-(methyloxy)-4-biphenylyl]-1H-pyrrolo[3,2-d]pyrimidine-2,4(3H,5H)-dione

To a solution of 5-(4-bromophenyl)-1H-pyrrolo[3,2-d]pyrimidine-2,4(3H,5H)-dione (Intermediate 57; 300 mg, 0.98 mmol) in 1,4-dioxane (2.5 mL)/water (1.5 mL) were added [2-fluoro-6-(methyloxy)phenyl]boronic acid (167 mg, 0.98 mmol), cesium carbonate (958 mg, 2.94 mmol), and palladium tetrakis (56.6 mg, 0.05 mmol). The reaction was performed under microwave irradiation at 160° C. for 15 min. Then the mixture was poured into DCM and washed with a 1N HCl solution. The organic layer was evaporated. The crude product was heated in methanol, filtered and the solid obtained was triturated in acetone, filtered, washed with diethyl ether, filtered and dried. The title compound 5-[2′-fluoro-6′-(methyloxy)-4-biphenylyl]-1H-pyrrolo[3,2-d]pyrimidine-2,4(3H,5H)-dione was obtained as a off-white solid (85 mg, 0.24 mmol, 24.69% yield).

LCMS: (M+H)⁺=352; Rt=2.72 min.

### Intermediate 61

### Ethyl 5-chloro-1-[2′-hydroxy-3′-(methyloxy)-4-biphenylyl]-3-{[({4-methyl-3-[(methyloxy)carbonyl]phenyl}amino)carbonyl]amino}-1H-pyrrole-2-carboxylate

To a solution of 4-methyl-3-[(methyloxy)carbonyl]benzoic acid (100 mg, 0.52 mmol) in toluene (20 mL) was added triethylamine (0.14 mL, 1.03 mmol) and diphenyl azidophosphate (0.13 mL, 0.62 mmol) were added. The reaction mixture was stirred hours at 90° C. After cooling, ethyl 3-amino-5-chloro-1-[2′-hydroxy-3′-(methyloxy)-4-biphenylyl]-1H-pyrrole-2-carboxylate (Intermediate 8; 200 mg, 0.52 mmol) was added and the reaction was stirred 3 days at 50° C. Water was added to the reaction and toluene was evaporated in vacuo. After extraction with DCM, drying on sodium sulfate and evaporation, the product was purified by chromatography on silica gel using cyclohexane/EtOAc 90/10 to 80/20 as gradient. The pure fractions were concentrated in vacuo to give the title compound ethyl 5-chloro-1-[2′-hydroxy-3′-(methyloxy)-4-biphenylyl]-3-{[({4-methyl-3-[(methyloxy)carbonyl]phenyl}amino)carbonyl]amino}-1H-pyrrole-2-carboxylate (100 mg, 0.17 mmol, 33.5% yield) as a white solid.

LCMS: (M+H)⁺=578, Rt=4.03 min.

The intermediates 62 and 63 were prepared by methods analogous to that described for intermediate 61.

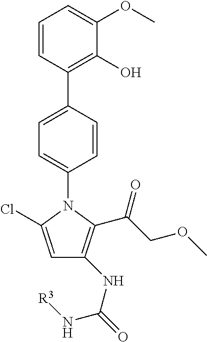

TABLE 8

| | | | | Physical |
|---|---|---|---|---|
| Intermediate | Name | Starting material | R³ | data |
| Intermediate 62 | Ethyl 5-chloro-1- [2′-hydroxy-3′- (methyloxy)-4- biphenylyl]-3- {[({3-(methyloxy)- 5- [(methyloxy) carbonyl]phenyl} amino)carbonyl] amino}-1H-pyrrole-2- carboxylate | Ethyl 3-amino-5- chloro-1-[2′-hydroxy- 3′-(methyloxy)-4- biphenylyl]-1H- pyrrole-2-carboxylate (Intermediate 8), and 3-(methyloxy)-5- [(methyloxy)carbonyl] benzoic acid | 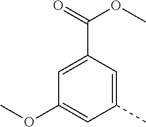 | LCMS: (M − H)⁺ = 592, Rt = 3.97 min. |
| Intermediate 63 | Ethyl 5-chloro-1- [2′-hydroxy-3′- (methyloxy)-4- biphenylyl]-3- {[({3-methyl-5- [(methyloxy) carbonyl]phenyl} amino)carbonyl] amino}-1H-pyrrole-2- carboxylate | Ethyl 3-amino-5- chloro-1-[2′-hydroxy- 3′-(methyloxy)-4- biphenylyl]-1H- pyrrole-2-carboxylate (Intermediate 8), and 3-methoxycarbonyl-5- methylbenzoic acid | 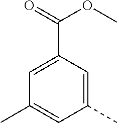 | LCMS: (M + H)⁺ = 578, Rt = 4.06 min. |

### Intermediate 64

### Ethyl 5-chloro-3-{[({3-[(ethyloxy)carbonyl]phenyl}amino)carbonyl]amino}-1-[2′-hydroxy-3′-(methyloxy)-4-biphenylyl]-1H-pyrrole-2-carboxylate

To a solution of ethyl 3-amino-5-chloro-1-[2′-hydroxy-3′-(methyloxy)-4-biphenylyl]-1H-pyrrole-2-carboxylate (Intermediate 8; 200 mg, 0.52 mmol) in toluene (50 mL) was added ethyl 3-isocyanatobenzoate (148 mg, 0.78 mmol) and the reaction was stirred at 80° C. for 18 hours. After cooling, the solvent was evaporated in vacuo to give the title compound ethyl 5-chloro-3-{[({3-[(ethyloxy)carbonyl]phenyl}amino)carbonyl]amino}-1-[2′-hydroxy-3′-(methyloxy)-4-biphenylyl]-1H-pyrrole-2-carboxylate (250 mg, 0.43 mmol, 84% yield) as a yellow oil.

LCMS: (M+H)⁺=578; Rt=4.29 min.

Intermediate 65 of formula (II) was prepared by methods analogous to that described for intermediate 64.

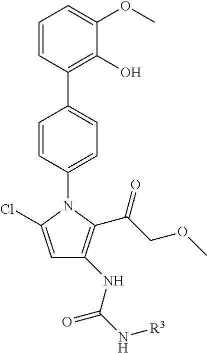

TABLE 9

| | | | | Physical |
|---|---|---|---|---|
| Intermediate | Name | Starting material | R³ | data |
| Intermediate 65 | Ethyl 5-chloro- 3-{[({4- [(ethyloxy) carbonyl]phenyl} amino)carbonyl] amino}-1-[2′- hydroxy-3′- (methyloxy)-4- biphenylyl]-1H- pyrrole-2- carboxylate | Ethyl 3-amino-5- chloro-1-[2′-hydroxy- 3′-(methyloxy)-4- biphenylyl]-1H- pyrrole-2- carboxylate (Intermediate 8) and ethyl 4- isocyanatobenzoate | 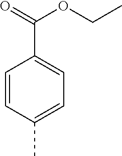 | LCMS: (M + H)⁺ = 578; Rt = 4.21 min. |

### Intermediate 66

### Ethyl 5-chloro-1-[2′-hydroxy-3′-(methyloxy)-4-biphenylyl]-3-[({[1-methyl-2-(methyloxy)-2-oxoethyl]amino}carbonyl)amino]-1H-pyrrole-2-carboxylate

To a solution of ethyl 3-amino-5-chloro-1-[2′-hydroxy-3′-(methyloxy)-4-biphenylyl]-1H-pyrrole-2-carboxylate (Intermediate 8; 200 mg, 0.52 mmol) in toluene (50 mL) was added methyl N-(oxomethylidene)alaninate (67 mg, 0.52 mmol) and the reaction was stirred at 80° C. 18 hours. After cooling, the solvent was evaporated in vacuo to give the title compound ethyl 5-chloro-1-[2′-hydroxy-3′-(methyloxy)-4-biphenylyl]-3-[({[1-methyl-2-(methyloxy)-2-oxoethyl]amino}carbonyl)amino]-1H-pyrrole-2-carboxylate (250 mg, 0.49 mmol, 94% yield) as an orange oil.

LCMS: (M+H)⁺=516; Rt=3.53 min.

### Intermediate 67

### (4-Chloro-2-hydroxyphenyl)boronic acid

To a solution of the commercially available [4-chloro-2-(methyloxy)phenyl]boronic acid (1.0 g, 5.36 mmol) in DCM was added boron tribromide (1M solution in DCM, 10.73 mL, 10.73 mmol) and the reaction was stirred at RT for 1 hour. The reaction was quenched by addition of ice, and the organic phase was separated. The aqueous phase was extracted with DCM, dried over sodium sulfate and evaporated in vacuo. The title compound, (4-chloro-2-hydroxyphenyl)boronic acid was obtained as a white powder (800 mg, 4.64 mmol, 87% yield).

LCMS: (M−H)⁺=171; Rt=4.40 min.

### Intermediate 68

### Ethyl 5-chloro-3-({[(2,4-dimethylphenyl)amino]carbonyl}amino)-1-[2′-hydroxy-3′-(methyloxy)-4-biphenylyl]-1H-pyrrole-2-carboxylate

The title compound was prepared by methods analogous to that described for Intermediate 20 using ethyl 3-amino-5-chloro-1-[2′-hydroxy-3′-(methyloxy)-4-biphenylyl]-1H-pyrrole-2-carboxylate (Intermediate 8) and 1-isocyanato-2,4-dimethylbenzene in toluene at 80° C.

LCMS: (M+H)⁺=534; Rt=3.97 min

### Intermediate 69

### Ethyl 1-[2′-hydroxy-3′-(methyloxy)-4-biphenylyl]-3-{[(3-pyridinylamino)carbonyl]amino}-1H-pyrrole-2-carboxylate

To a solution of carbonyl diimidazole (103 mg, 0.64 mmol) in DCM was added a solution of 3-pyridinamine (50 mg, 0.53 mmol) in DCM. The reaction was stirred at RT for 3 hours. Then ethyl 3-amino-1-[2′-hydroxy-3′-(methyloxy)-4-biphenylyl]-1H-pyrrole-2-carboxylate hydrochloride (Intermediate 4; 150 mg, 0.43 mmol) in DMF (3 mL) was added and the mixture was stirred at 80° C. overnight. The solvent was evaporated in vacuo and the crude title compound was purified by chromatography using DCM/MeOH 100/0 to 90/10 to give the title compound ethyl 1-[2′-hydroxy-3′-(methyloxy)-4-biphenylyl]-3-{[(3-pyridinylamino)carbonyl]amino}-1H-pyrrole-2-carboxylate (40 mg, 0.09 mmol, 15.94% yield) as a brown oil.

LCMS: (M+H)⁺=473; Rt=3.36 min.

### Intermediate 70

### 5-[2′-Hydroxy-3′-(methyloxy)-4-biphenylyl]-3-(3-pyridinyl)-1H-pyrrolo[3,2-d]pyrimidine-2,4(3H,5H)-dione

To a solution of ethyl 1-[2′-hydroxy-3′-(methyloxy)-4-biphenylyl]-3-{[(3-pyridinylamino)carbonyl]amino}-1H-pyrrole-2-carboxylate (Intermediate 69; 40 mg, 0.09 mmol) in ethanol (5 mL) was added a solution of sodium (3.89 mg, 0.17 mmol) in ethanol (5 mL). The reaction was stirred at 80° C. overnight. The reaction mixture was concentrated in vacuo, taken in water. Then acetic acid was added until precipitation. The product was filtered, dried and purified by chromatography using DCM/MeOH 100/0 to 90/10 as gradient. The appropriate fractions were combined and concentrated in vacuo to give the title compound 5-[2′-hydroxy-3′-(methyloxy)-4-biphenylyl]-3-(3-pyridinyl)-1H-pyrrolo[3,2-d]pyrimidine-2,4(3H,5H)-dione as a brown powder (5 mg, 0.01 mmol, 13.85% yield).

LCMS: (M+H)⁺=427; Rt=2.71 min.

HRMS: calculated for C₂₄H₁₉N₄O₄ (M+H)+: 427.1406; found: 427.1432. Rt: 2.46 min.

### Example 1

### 3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid

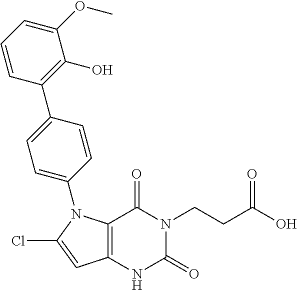 [SMILES: COc1cccc(-c2ccc(-n3c(Cl)cc4[nH]c(=O)n(CCC(=O)O)c(=O)c43)cc2)c1O]

To a solution of ethyl 5-chloro-3-[({[3-(ethyloxy)-3-oxopropyl]amino}carbonyl)amino]-1-[2′-hydroxy-3′-(methyloxy)-4-biphenylyl]-1H-pyrrole-2-carboxylate (Intermediate 12 1.08 g, 2.04 mmol) in ethanol (50 mL) was added sodium (0.19 g, 8.15 mmol). The reaction was stirred at 90° C. for 2 hours. After cooling, the reaction was concentrated in vacuo and acidified with a 0.5N H₂SO₄ solution. The compound was filtered and washed with water. The solid was dissolved in 1N NaOH solution, extracted with DCM and the basic phase was acidified with concentrated HCl. The precipitate was filtered, washed with water and dried. Recrystallisation from ethanol gave the title compound 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid (430 mg, 0.94 mmol, 46.3% yield) as a cream solid.

LCMS: (M+H)⁺=456; Rt=2.56 min

HRMS: calculated for C₂₂H₁₉ClN₃O₆ (M+H)⁺: 456.0962; found: 456.0946; Rt=2.28 min.

Examples 2 to 4 of the general formula below were prepared by methods analogous to that described for Example 1.

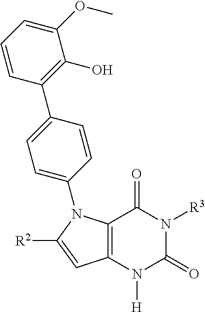

TABLE 10

| | | Starting | | | Physical |
|---|---|---|---|---|---|
| Example | Name | Material | R² | R³ | data |
| Example 2 | 4-{6-Chloro- 5-[2′- hydroxy-3′- (methyloxy)- 4- biphenylyl]- 2,4-dioxo- 1,2,4,5- tetrahydro- 3H- pyrrolo[3,2- d]pyrimidin- 3-yl}butanoic acid | Ethyl 5-chloro- 3-[({[4- (ethyloxy)-4- oxobutyl]amino} carbonyl)amino]- 1-[2′-hydroxy- 3′-(methyloxy)- 4-biphenylyl]- 1H-pyrrole-2- carboxylate (Intermediate 13) | Cl | 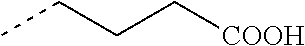 | LCMS: (M + H)⁺ = 470; Rt = 2.60 min. HRMS: calculated for C₂₃H₂₁ClN₃O₆ (M + H)⁺: 470.1119; found: 470.1115; Rt = 2.35 min. |
| Example 3 | {6-Chloro-5- [2′-hydroxy- 3′- (methyloxy)- 4- biphenylyl]- 2,4-dioxo- 1,2,4,5- tetrahydro- 3H- pyrrolo[3,2- d]pyrimidin- 3-yl}acetic acid | Ethyl 5-chloro- 3-[({[2- (ethyloxy)-2- oxoethyl]amino} carbonyl)amino]- 1-[2′-hydroxy- 3′-(methyloxy)- 4-biphenylyl]- 1H-pyrrole-2- carboxylate (Intermediate 14) | Cl | 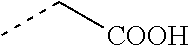 | LCMS: (M + H)⁺ = 442; Rt = 2.37 min. HRMS: calculated for C₂₁H₁₇ClN₃O₆ (M + H)⁺: 442.0806; found: 442.0802; Rt = 2.21 min. |
| Example 4 | 3-{5-[2′- Hydroxy-3′- (methyloxy)- 4- biphenylyl]- 2,4-dioxo- 1,2,4,5- tetrahydro- 3H- pyrrolo[3,2- d]pyrimidin- 3- yl}propanoic acid | Ethyl 3-[({[3- (ethyloxy)-3- oxopropyl]amino} carbonyl)amino]- 1-[2′- hydroxy-3′- (methyloxy)-4- biphenylyl]-1H- pyrrole-2- carboxylate (Intermediate 15) | H | 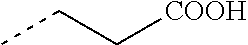 | LCMS: (M + H)⁺ = 422; Rt = 2.29 min. HRMS: calculated for C₂₂H₂₀N₃O₆ (M + H)⁺: 422.1352; found: 422.1379; Rt = 2.14 min. |

Examples 5 and 6 of the general formula below were prepared by methods analogous to that described for Example 1.

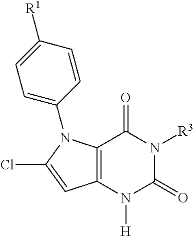

TABLE 11

| | | | | | Physical |
|---|---|---|---|---|---|
| Example | Name | Starting material | R¹ | R³ | data |
| Example 5 | 3-[6-Chloro-5- (2′-hydroxy-3′- methyl-4- biphenylyl)-2,4- dioxo-1,2,4,5- tetrahydro-3H- pyrrolo[3,2- d]pyrimidin-3- yl]propanoic acid | Ethyl 5-chloro-3- [({[3-(ethyloxy)-3- oxopropyl]amino} carbonyl)amino]-1- (2′-hydroxy-3′- methyl-4- biphenylyl)-1H- pyrrole-2- carboxylate (Intermediate 16) | 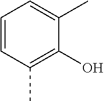 | 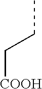 | LCMS: (M + H)⁺ = 440; Rt = 2.58 min. HRMS: calculated for C₂₂H₁₉ClN₃ O₅ (M + H)⁺: 440.1013; found: 440.1016. Rt = 2.40 min. |
| Example 6 | 3-[6-Chloro-5- (2′-hydroxy-4- biphenylyl)-2,4- dioxo-1,2,4,5- tetrahydro-3H- pyrrolo[3,2- d]pyrimidin-3- yl]propanoic acid | Ethyl 5-chloro-3- [({[3-(ethyloxy)-3- oxopropyl]amino} carbonyl)amino]-1- (2′-hydroxy-4- biphenylyl)-1H- pyrrole-2- carboxylate (Intermediate 36) | 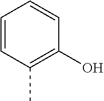 | 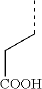 | LCMS: (M + H)⁺ = 426; Rt = 2.38 min HRMS: calculated for C₂₁H₁₇ClN₃ O₅ (M + H)⁺: 426.0857; found: 426.0857. Rt = 2.18 min. |

### Example 7

### 6-Chloro-5-[2′-hydroxy-3′-(methyloxy)-4-biphenylyl]-3-phenyl-1H-pyrrolo[3,2-d]pyrimidine-2,4(3H,5H)-dione

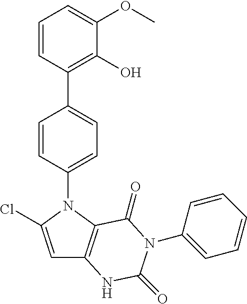 [SMILES: COc1cccc(-c2ccc(-n3c(Cl)cc4[nH]c(=O)n(-c5ccccc5)c(=O)c43)cc2)c1O]

A solution of sodium (8.86 mg, 0.39 mmol) in ethanol (5 mL) was added to a solution of ethyl 5-chloro-1-[2′-hydroxy-3′-(methyloxy)-4-biphenylyl]-3-{[(phenylamino)carbonyl]amino}-1H-pyrrole-2-carboxylate (Intermediate 20; 78 mg, 0.15 mmol) in ethanol (20 mL).

The mixture was heated at reflux temperature for 1 h 30 mins before being cooled acidified with acetic acid and concentrated in vacuo. The residue was washed with water and with DCM. The solid was recrystallised from acetonitrile to give the title compound, 6-chloro-5-[2′-hydroxy-3′-(methyloxy)-4-biphenylyl]-3-phenyl-1H-pyrrolo[3,2-d]pyrimidine-2,4(3H,5H)-dione (46 mg, 0.1 mmol, 64.9% yield) as an off-white solid.

LCMS: (M+H)⁺=460; Rt=3.19 min.

HRMS: calculated for C₂₅H₁₉ClN₃O₄ (M+H)⁺: 460.1064; found: 460.1088. Rt=2.91 min.

Examples 8 to 40 of the general formula below were prepared by methods analogous to that described for Example 7.

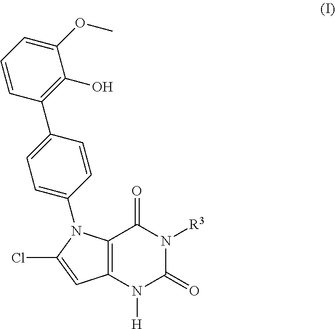

TABLE 12

| | | | | Physical |
|---|---|---|---|---|
| Example | Name | Starting material | R³ | data |
| Example 8 | 6-Chloro-3-(2,6- dichlorophenyl)- 5-[2′-hydroxy-3′- (methyloxy)-4- biphenylyl]-1H- pyrrolo[3,2- d]pyrimidine- 2,4(3H,5H)- dione | Ethyl 5-chloro-3- ({[(2,6- dichlorophenyl)amino] carbonyl}amino)-1-[2′- hydroxy-3′- (methyloxy)-4- biphenylyl]-1H- pyrrole-2-carboxylate (Intermediate 37) | 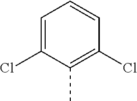 | LCMS: (M + H)⁺ = 528 530; Rt = 3.43 min. HRMS: calculated for C₂₅H₁₇Cl₃N₃O₄ (M + H)⁺: 528.0284; found: 528.0281. Rt = 3.04 min. |
| Example 9 | 6-Chloro-3-(3- chloro-2- fluorophenyl)-5- [2′-hydroxy-3′- (methyloxy)-4- biphenylyl]-1H- pyrrolo[3,2- d]pyrimidine- 2,4(3H,5H)- dione | Ethyl 5-chloro-3-({[(3- chloro-2- fluorophenyl)amino] carbonyl}amino)-1-[2′- hydroxy-3′- (methyloxy)-4- biphenylyl]-1H- pyrrole-2-carboxylate (Intermediate 38) | 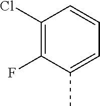 | LCMS: (M + H)⁺ = 512; Rt = 3.43 min. HRMS: calculated for C₂₅H₁₇Cl₂FN₃O₄ (M + H)⁺: 512.0580; found: 512.0554. Rt = 3.04 min. |
| Example 10 | 6-Chloro-3-(3- fluoro-2- methylphenyl)- 5-[2′-hydroxy-3′- (methyloxy)-4- biphenylyl]-1H- pyrrolo[3,2- d]pyrimidine- 2,4(3H,5H)- dione | Ethyl 5-chloro-3-({[(3- fluoro-2- methylphenyl)amino] carbonyl}amino)-1-[2′- hydroxy-3′- (methyloxy)-4- biphenylyl]-1H- pyrrole-2-carboxylate (Intermediate 39) | 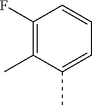 | LCMS: (M + H)⁺ = 492; Rt = 3.37 min. HRMS: calculated for C₂₆H₂₀ClFN₃O₄ (M + H)⁺: 492.1126; found: 492.1106. Rt = 2.97 min. |
| Example 11 | 6-Chloro-3-(2,3- dimethylphenyl)- 5-[2′-hydroxy- 3′-(methyloxy)- 4-biphenylyl]- 1H-pyrrolo[3,2- d]pyrimidine- 2,4(3H,5H)- dione | Ethyl 5-chloro-3- ({[(2,3- dimethylphenyl)amino] carbonyl}amino)-1- [2′-hydroxy-3′- (methyloxy)-4- biphenylyl]-1H- pyrrole-2-carboxylate (Intermediate 40) | 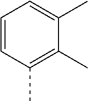 | LCMS: (M + H)⁺ = 488, Rt = 3.37 min HRMS: calculated for C₂₇H₂₃ClN₃O₄ (M + H)⁺: 488.1377; found: 488.1395 Rt = 2.99 min. |
| Example 12 | 3-{6-Chloro-5- [2′-hydroxy-3′- (methyloxy)-4- biphenylyl]-2,4- dioxo-1,2,4,5- tetrahydro-3H- pyrrolo[3,2- d]pyrimidin-3- yl}benzonitrile | Ethyl 5-chloro-3-({[(3- cyanophenyl)amino] carbonyl}amino)-1-[2′- hydroxy-3′- (methyloxy)-4- biphenylyl]-1H- pyrrole-2-carboxylate (Intermediate 41) | 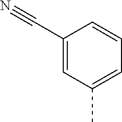 | LCMS: (M + H)⁺ = 485; Rt = 3.22 min. HRMS: calculated for C₂₆H₁₈ClN₄O₄ (M + H)⁺: 485.1017; found: 485.1008 Rt = 2.86 min. |
| Example 13 | 6-Chloro-3-(3- chloro-2- methylphenyl)- 5-[2′-hydroxy-3′- (methyloxy)-4- biphenylyl]-1H- pyrrolo[3,2- d]pyrimidine- 2,4(3H,5H)- dione | Ethyl 5-chloro-3-({[(3- chloro-2- methylphenyl)amino] carbonyl}amino)-1-[2′- hydroxy-3′- (methyloxy)-4- biphenylyl]-1H- pyrrole-2-carboxylate (Intermediate 42) | 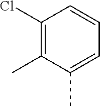 | LCMS: (M + H)⁺ = 508, Rt = 3.45 min HRMS: calculated for C₂₆H₂₀Cl₂N₃O₄ (M + H)⁺: 508.0831; found: 508.0846 Rt = 3.00 min. |
| Example 14 | 6-Chloro-3-(2- chloro-6- methylphenyl)- 5-[2′-hydroxy-3′- (methyloxy)-4- biphenylyl]-1H- pyrrolo[3,2- d]pyrimidine- 2,4(3H,5H)- dione | Ethyl 5-chloro-3-({[(2- chloro-6- methylphenyl)amino] carbonyl}amino)-1-[2′- hydroxy-3′- (methyloxy)-4- biphenylyl]-1H- pyrrole-2-carboxylate (Intermediate 43) | 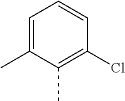 | LCMS: (M + H)⁺ = 508, Rt = 3.39 min HRMS: calculated for C₂₆H₂₀Cl₂N₃O₄ (M + H)⁺: 508.0831; found: 508.0830 Rt = 2.94 min. |
| Example 15 | 6-Chloro-3-(2,6- difluorophenyl)- 5-[2′-hydroxy-3′- (methyloxy)-4- biphenylyl]-1H- pyrrolo[3,2- d]pyrimidine- 2,4(3H,5H)- dione | Ethyl 5-chloro-3- ({[(2,6- difluorophenyl)amino] carbonyl}amino)-1-[2′- hydroxy-3′- (methyloxy)-4- biphenylyl]-1H- pyrrole-2-carboxylate (Intermediate 44) | 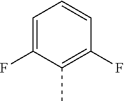 | LCMS: (M + H)⁺ = 496, Rt = 3.30 min HRMS: calculated for C₂₅H₁₇ClF₂N₃O₄ (M + H)⁺: 496.0876; found: 496.0826 Rt = 2.90 min . |
| Example 16 | 6-Chloro-5-[2′- hydroxy-3′- (methyloxy)-4- biphenylyl]-3-[3- (trifluoromethyl) phenyl]-1H- pyrrolo[3,2- d]pyrimidine- 2,4(3H,5H)- dione | Ethyl 5-chloro-1-[2′- hydroxy-3′- (methyloxy)-4- biphenylyl]-3-[({[3- (trifluoromethyl)phenyl] amino}carbonyl)amino]- 1H-pyrrole-2- carboxylate (Intermediate 45) | 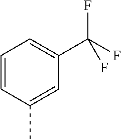 | LCMS: (M + H)⁺ = 528, Rt = 3.55 min HRMS: calculated for C₂₆H₁₈ClF₃N₃O₄ (M + H)⁺: 528.0938; found: 528.0891 Rt = 2.96 min. |
| Example 17 | 6-Chloro-3-(2- hydroxyethyl)-5- [2′-hydroxy-3′- (methyloxy)-4- biphenylyl]-1H- pyrrolo[3,2- d]pyrimidine- 2,4(3H,5H)- dione | Ethyl 5-chloro-1-[2′- hydroxy-3′- (methyloxy)-4- biphenylyl]-3-{[({2-[(2- methylacryloyl)oxy] ethyl}amino)carbonyl] amino}-1H-pyrrole-2- carboxylate (Intermediate 46) | 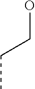 | LCMS: (M + H)⁺ = 428, Rt = 2.87 min HRMS: calculated for C₂₁H₁₉ClN₃O₅ (M + H)⁺: 428.1013; found: 428.1037 Rt = 2.43 min. |
| Example 18 | 6-Chloro-5-[2′- hydroxy-3′- (methyloxy)-4- biphenylyl]-3-[2- (methyloxy)ethyl]- 1H-pyrrolo[3,2- d]pyrimidine- 2,4(3H,5H)- dione | Ethyl 5-chloro-1-[2′- hydroxy-3′- (methyloxy)-4- biphenylyl]-3-[({[2- (methyloxy)ethyl]amino} carbonyl)amino]-1H- pyrrole-2-carboxylate (Intermediate 47) | 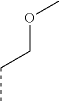 | LCMS: (M + H)⁺ = 442, Rt = 3.11 min HRMS: calculated for C₂₂H₂₁ClN₃O₅ (M + H)⁺: 442.1170; found: 442.1177 Rt = 2.66 min. |
| Example 19 | 6-Chloro-5-[2′- hydroxy-3′- (methyloxy)-4- biphenylyl]-3-(2- methylphenyl)- 1H-pyrrolo[3,2- d]pyrimidine- 2,4(3H,5H)- dione | Ethyl 5-chloro-1-[2′- hydroxy-3′- (methyloxy)-4- biphenylyl]-3-({[(2- methylphenyl)amino] carbonyl}amino)-1H- pyrrole-2-carboxylate (Intermediate 21) | 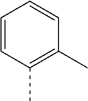 | LCMS: (M + H)⁺ = 474, Rt = 3.29 min HRMS: calculated for C₂₆H₂₁ClN₃O₄ (M + H)⁺: 474.1220; found: 474.1231 Rt = 2.94 min. |
| Example 20 | 6-Chloro-3-(2- chlorophenyl)-5- [2′-hydroxy-3′- (methyloxy)-4- biphenylyl]-1H- pyrrolo[3,2- d]pyrimidine- 2,4(3H,5H)- dione | Ethyl 5-chloro-3-({[(2- chlorophenyl)amino] carbonyl}amino)-1-[2′- hydroxy-3′- (methyloxy)-4- biphenylyl]-1H- pyrrole-2-carboxylate (Intermediate 22) | 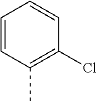 | LCMS: (M + H)⁺ = 494, Rt = 3.29 min HRMS: calculated for C₂₅H₁₈Cl₂N₃O₄ (M + H)⁺: 494.0674; found: 494.0638 Rt = 2.92 min. |
| Example 21 | 6-Chloro-3-(3- chlorophenyl)-5- [2′-hydroxy-3′- (methyloxy)-4- biphenylyl]-1H- pyrrolo[3,2- d]pyrimidine- 2,4(3H,5H)- dione | Ethyl 5-chloro-3-({[(3- chlorophenyl)amino] carbonyl}amino)-1-[2′- hydroxy-3′- (methyloxy)-4- biphenylyl]-1H- pyrrole-2-carboxylate (Intermediate 23) | 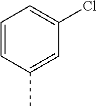 | LCMS: (M + H)⁺ = 494, Rt = 3.38 min HRMS: calculated for C₂₅H₁₈Cl₂N₃O₄ (M + H)⁺: 494.0674; found: 494.0647 Rt = 3.02 min. |
| Example 22 | 6-chloro-3-(2- fluorophenyl)-5- [2′-hydroxy-3′- (methyloxy)-4- biphenylyl]-1H- pyrrolo[3,2- d]pyrimidine- 2,4(3H,5H)- dione | Ethyl 5-chloro-3-({[(2- fluorophenyl)amino] carbonyl}amino)-1-[2′- hydroxy-3′- (methyloxy)-4- biphenylyl]-1H- pyrrole-2-carboxylate (Intermediate 24) | 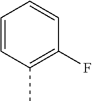 | LCMS: (M + H)⁺ = 478, Rt = 3.25 min HRMS: calculated for C₂₅H₁₈ClFN₃O₄ (M + H)⁺: 478.0970; found: 478.0977 Rt = 2.90 min. |
| Example 23 | 6-Chloro-5-[2′- hydroxy-3′- (methyloxy)-4- biphenylyl]-3-[3- (methyloxy) phenyl]-1H- pyrrolo[3,2- d]pyrimidine- 2,4(3H,5H)- dione | Ethyl 5-chloro-1-[2′- hydroxy-3′- (methyloxy)-4- biphenylyl]-3-[({[3- (methyloxy)phenyl] amino}carbonyl)amino]- 1H-pyrrole-2- carboxylate (Intermediate 25) | 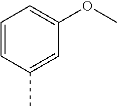 | LCMS: (M + H)⁺ = 490, Rt = 3.22 min HRMS: calculated for C₂₆H₂₁ClN₃O₅ (M + H)⁺: 490.1170; found: 490.1168 Rt = 2.87 min. |
| Example 24 | 6-Chloro-5-[2′- hydroxy-3′- (methyloxy)-4- biphenylyl]-3-[4- (methyloxy) phenyl]-1H- pyrrolo[3,2- d]pyrimidine- 2,4(3H,5H)- dione | Ethyl 5-chloro-1-[2′- hydroxy-3′- (methyloxy)-4- biphenylyl]-3-[({[4- (methyloxy)phenyl] amino}carbonyl)amino]- 1H-pyrrole-2- carboxylate (Intermediate 26) | 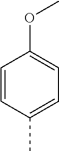 | LCMS: (M + H)⁺ = 490, Rt = 3.21 min HRMS: calculated for C₂₆H₂₁ClN₃O₅ (M + H)⁺: 490.1170; found: 490.1146 Rt = 2.94 min. |
| Example 25 | 6-Chloro-3-(4- fluorophenyl)-5- [2′-hydroxy-3′- (methyloxy)-4- biphenylyl]-1H- pyrrolo[3,2- d]pyrimidine- 2,4(3H,5H)- dione | Ethyl 5-chloro-3-({[(4- fluorophenyl)amino] carbonyl}amino)-1-[2′- hydroxy-3′- (methyloxy)-4- biphenylyl]-1H- pyrrole-2-carboxylate (Intermediate 27) | 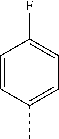 | LCMS: (M + H)⁺ = 478, Rt = 3.25 min HRMS: calculated for C₂₅H₁₈ClFN₃O₄ (M + H)⁺: 478.0970; found: 478.0953 Rt = 2.97 min. |
| Example 26 | 6-Chloro-3-(4- chlorophenyl)-5- [2′-hydroxy-3′- (methyloxy)-4- biphenylyl]-1H- pyrrolo[3,2- d]pyrimidine- 2,4(3H,5H)- dione | Ethyl 5-chloro-3-({[(4- chlorophenyl)amino] carbonyl}amino)-1-[2′- hydroxy-3′- (methyloxy)-4- biphenylyl]-1H- pyrrole-2-carboxylate (Intermediate 28) | 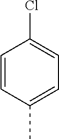 | LCMS: (M + H)⁺ = 494, Rt = 3.38 min HRMS: calculated for C₂₅H₁₈Cl₂N₃O₄ (M + H)⁺: 494.0674; found: 494.0676 Rt = 3.07 min . |
| Example 27 | 6-Chloro-5-[2′- hydroxy-3′- (methyloxy)-4- biphenylyl]-3-(4- methylphenyl)- 1H-pyrrolo[3,2- d]pyrimidine- 2,4(3H,5H)- dione | Ethyl 5-chloro-1-[2′- hydroxy-3′- (methyloxy)-4- biphenylyl]-3-({[(4- methylphenyl)amino] carbonyl}amino)-1H- pyrrole-2-carboxylate (Intermediate 29) | 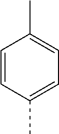 | LCMS: (M + H)⁺ = 474, Rt = 3.31 min HRMS: calculated for C₂₆H₂₁ClN₃O₄ (M + H)⁺: 474.1220; found: 474.1248 Rt = 2.99 min. |
| Example 28 | 6-Chloro-5-[2′- hydroxy-3′- (methyloxy)-4- biphenylyl]-3-(3- methylphenyl)- 1H-pyrrolo[3,2- d]pyrimidine- 2,4(3H,5H)- dione | Ethyl 5-chloro-1-[2′- hydroxy-3′- (methyloxy)-4- biphenylyl]-3-({[(3- methylphenyl)amino] carbonyl}amino)-1H- pyrrole-2-carboxylate (Intermediate 30) | 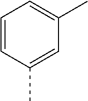 | LCMS: (M + H)⁺ = 474, Rt = 3.32 min HRMS: calculated for C₂₆H₂₁ClN₃O₄ (M + H)⁺: 474.1220; found: 474.1191 Rt = 2.99 min. |
| Example 29 | 4-{6-Chloro-5- [2′-hydroxy-3′- (methyloxy)-4- biphenylyl]-2,4- dioxo-1,2,4,5- tetrahydro-3H- pyrrolo[3,2- d]pyrimidin-3- yl}benzonitrile | Ethyl 5-chloro-3-({[(4- cyanophenyl)amino] carbonyl}amino)-1-[2′- hydroxy-3′- (methyloxy)-4- biphenylyl]-1H- pyrrole-2-carboxylate (Intermediate 31) | 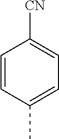 | LCMS: (M + H)⁺ = 485; Rt = 3.18 min ¹H NMR: (DMSO-d⁶, 300 MHz) δ 7.90 (d, 2H), 7.62 (d, 2H), 7.48 (d, 2H), 7.39 (d, 2H), 6.93 (m, 3H), 6.32 (s, 1H), 3.85 (s, 3H). |
| Example 30 | 6-Chloro-5-[2′- hydroxy-3′- (methyloxy)-4- biphenylyl]-3-(2- methyl-6- nitrophenyl)-1H- pyrrolo[3,2- d]pyrimidine- 2,4(3H,5H)- dione | Ethyl 5-chloro-1-[2′- hydroxy-3′- (methyloxy)-4- biphenylyl]-3-({[(2- methyl-6- nitrophenyl)amino] carbonyl}amino)-1H- pyrrole-2-carboxylate (Intermediate 32) | 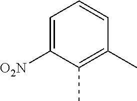 | LCMS: (M + H)⁺ = 519, Rt = 3.34 min. HRMS: calculated for C₂₆H₂₀ClN₄O₆ (M + H)⁺: 519.1071; found: 519.1071 Rt = 2.95 min. |
| Example 31 | 6-Chloro-3-[2- fluoro-3- (trifluoromethyl) phenyl]-5-[2′- hydroxy-3′- (methyloxy)-4- biphenylyl]-1H- pyrrolo[3,2- d]pyrimidine- 2,4(3H,5H)- dione | Ethyl 5-chloro-3-[({[2- fluoro-3- (trifluoromethyl)phenyl] amino}carbonyl)amino]- 1-[2′-hydroxy-3′- (methyloxy)-4- biphenylyl]-1H- pyrrole-2-carboxylate (Intermediate 33) | 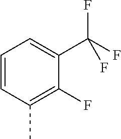 | LCMS: (M + H)⁺ = 546; Rt = 3.53 min HRMS: calculated for C₂₆H₁₇ClF₄N₃O₄ (M + H)⁺: 546.0844; found: 546.0850 Rt = 3.13 min. |
| Example 32 | 6-Chloro-3-(2,6- dimethylphenyl)- 5-[2′-hydroxy- 3′-(methyloxy)- 4-biphenylyl]- 1H-pyrrolo[3,2- d]pyrimidine- 2,4(3H,5H)- dione | Ethyl 5-chloro-3- ({[(2,6- dimethylphenyl)amino] carbonyl}amino)-1- [2′-hydroxy-3′- (methyloxy)-4- biphenylyl]-1H- pyrrole-2-carboxylate (Intermediate 34) | 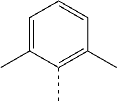 | LCMS: (M + H)⁺ = 488; Rt = 3.36 min HRMS: calculated for C₂₇H₂₃ClN₃O₄ (M + H)⁺: 488.1377; found: 488.1391 Rt = 3.00 min. |
| Example 33 | 6-Chloro-3-(2,3- dichlorophenyl)- 5-[2′-hydroxy-3′- (methyloxy)-4- biphenylyl]-1H- pyrrolo[3,2- d]pyrimidine- 2,4(3H,5H)- dione | Ethyl 5-chloro-3- ({[(2,3- dichlorophenyl)amino] carbonyl}amino)-1-[2′- hydroxy-3′- (methyloxy)-4- biphenylyl]-1H- pyrrole-2-carboxylate (Intermediate 48) | 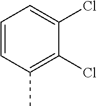 | LCMS: (M + H)⁺ = 528-530; Rt = 3.47 min HRMS: calculated for C₂₅H₁₇Cl₃N₃O₄ (M + H)⁺: 528.0284; found: 528.0321 Rt = 3.09 min. |
| Example 34 | 6-Chloro-5-[2′- hydroxy-3′- (methyloxy)-4- biphenylyl]-3-(2- methyl-3- nitrophenyl)-1H- pyrrolo[3,2- d]pyrimidine- 2,4(3H,5H)- dione | Ethyl 5-chloro-1-[2′- hydroxy-3′- (methyloxy)-4- biphenylyl]-3-({[(2- methyl-3- nitrophenyl)amino] carbonyl}amino)-1H- pyrrole-2-carboxylate (Intermediate 35) | 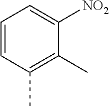 | LCMS: (M + H)⁺ = 519; Rt = 3.34 min HRMS: calculated for C₂₆H₂₀ClN₄O₆ (M + H)⁺: 519.1071; found: 519.1036 Rt = 2.96 min. |
| Example 35 | 6-Chloro-5-[2′- hydroxy-3′- (methyloxy)-4- biphenylyl]-3-[3- (methyloxy) propyl]-1H- pyrrolo[3,2- d]pyrimidine- 2,4(3H,5H)- dione | Ethyl 5-chloro-1-[2′- hydroxy-3′- (methyloxy)-4- biphenylyl]-3-[({[3- (methyloxy)propyl] amino}carbonyl)amino]- 1H-pyrrole-2- carboxylate (Intermediate 49) | 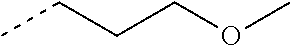 | LCMS: (M + H)⁺ = 456 Rt = 3.06 min ¹H NMR: (DMSO-d⁶, 300 MHz) δ 11.35(s, 1H), 8.79 (s, 1H), 7.65 (d, 2H), 7.37 (d, 2H), 6.95 (m, 3H), 6.22 (s, 1H), 3.87 (s, 3H), 3.8 (t, 3H), 3.30 (m, 2H), 3.18 (s, 3H), 1.70 (m, 2H). |
| Example 36 | 3-[3,4- Bis(methyloxy) phenyl]-6-chloro- 5-[2′-hydroxy-3′- (methyloxy)-4- biphenylyl]-1H- pyrrolo[3,2- d]pyrimidine- 2,4(3H,5H)- dione | Ethyl 3-[({[3,4- bis(methyloxy)phenyl] amino}carbonyl)amino]- 5-chloro-1-[2′- hydroxy-3′- (methyloxy)-4- biphenylyl]-1H- pyrrole-2-carboxylate (Intermediate 50) | 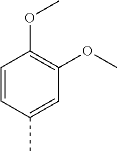 | LCMS: (M + H)⁺ = 520; Rt = 3.03 min HRMS: calculated for C₂₇H₂₃ClN₃O₆ (M + H)⁺: 520.1275; found: 520.1243 Rt = 2.74 min . |
| Example 37 | 3-[3,5- Bis(methyloxy) phenyl]-6-chloro- 5-[2′-hydroxy-3′- (methyloxy)-4- biphenylyl]-1H- pyrrolo[3,2- d]pyrimidine- 2,4(3H,5H)- dione | Ethyl 3-[({[3,5- bis(methyloxy)phenyl] amino}carbonyl)amino]- 5-chloro-1-[2′-hydroxy- 3′-(methyloxy)-4- biphenylyl]-1H- pyrrole-2-carboxylate (Intermediate 51) | 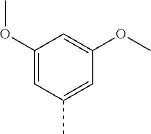 | LCMS: (M + H)⁺ = 520; Rt = 3.18 min HRMS: calculated for C₂₇H₂₃ClN₃O₆ (M + H)⁺: 520.1275; found: 520.1245 Rt = 2.87 min . |
| Example 38 | 6-Chloro-3-(2,4- dimethylphenyl)- 5-[2′-hydroxy- 3′-(methyloxy)- 4-biphenylyl]- 1H-pyrrolo[3,2- d]pyrimidine- 2,4(3H,5H)- dione | Ethyl 5-chloro-3- ({[(2,4- dimethylphenyl)amino] carbonyl}amino)-1- [2′-hydroxy-3′- (methyloxy)-4- biphenylyl]-1H- pyrrole-2-carboxylate (Intermediate 68) | 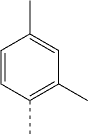 | LCMS: (M + H)⁺ = 488; Rt = 3.33 min HRMS: calculated for C₂₇H₂₃ClN₃O₄ (M + H)⁺: 488.1317; found: 488.1412 Rt = 2.98 min . |
| Example 39 | 6-Chloro-5-[2′- hydroxy-3′- (methyloxy)-4- biphenylyl]-3-(2- naphthalenyl)- 1H-pyrrolo[3,2- d]pyrimidine- 2,4(3H,5H)- dione | Ethyl 5-chloro-1-[2′- hydroxy-3′- (methyloxy)-4- biphenylyl]-3-{[(2- naphthalenylamino) carbonyl]amino}-1H- pyrrole-2-carboxylate (Intermediate 53) | 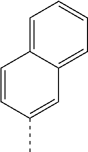 | LCMS: (M + H)⁺ = 510; Rt = 3.37 min HRMS: calculated for C₂₉H₂₁ClN₃O₄ (M + H)⁺: 510.1220; found: 510.1195 Rt = 3.05 min |
| Example 40 | 6-Chloro-5-[2′- hydroxy-3′- (methyloxy)-4- biphenylyl]-3-[2- (methyloxy) phenyl]-1H- pyrrolo[3,2- d]pyrimidine- 2,4(3H,5H)- dione | Ethyl 5-chloro-1-[2′- hydroxy-3′- (methyloxy)-4- biphenylyl]-3-[({[2- (methyloxy)phenyl] amino}carbonyl) amino]- 1H-pyrrole-2- carboxylate (Intermediate 54) | 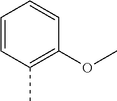 | LCMS: (M + H)⁺ = 490; Rt = 3.12 min HRMS: calculated for. C₂₆H₂₁ClN₃O₅ (M + H)⁺: 490.1170; found: 490.1172 Rt = 2.82 min |

### Example 41

### 5-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-2-methylbenzoic acid

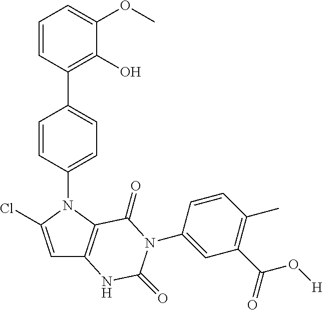 [SMILES: COc1cccc(-c2ccc(-n3c(Cl)cc4[nH]c(=O)n(-c5ccc(C)c(C(=O)O)c5)c(=O)c43)cc2)c1O]

To a solution of ethyl 5-chloro-1-[2′-hydroxy-3′-(methyloxy)-4-biphenylyl]-3-{[({4-methyl-3-[(methyloxy)carbonyl]phenyl}amino)carbonyl]amino}-1H-pyrrole-2-carboxylate (Intermediate 61; 100 mg, 0.17 mmol) in ethanol (40 mL) was added a 1N solution of sodium hydroxide (0.69 mL, 0.69 mmol), the reaction was stirred overnight at 90° C. After cooling, the reaction was concentrated in vacuo and acidified with a 1N HCl solution. The precipitate was filtered, washed with water, dried and recrystallized from ethanol to give the title compound, 5-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-2-methylbenzoic acid (40 mg, 0.08 mmol, 44.6% yield).

LCMS: (M+H)⁺=518; Rt=2.68 min

HRMS: calculated for C₂₇H₂₁ClN₃O₆(M+H)⁺:518.1119; found: 518.1110 Rt=2.39 min

Examples 42 to 46 of the general formula below were prepared by methods analogous to that described for Example 41.

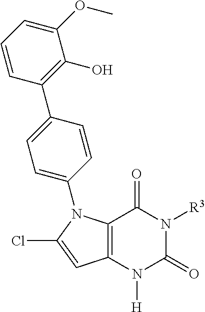

TABLE 13

| Example | Name | Starting material | R³ | Physical data |
|---|---|---|---|---|
| Example 42 | 3-{6-Chloro-5-[2′- hydroxy-3′- (methyloxy)-4- biphenylyl]-2,4- dioxo-1,2,4,5- tetrahydro-3H- pyrrolo[3,2- d]pyrimidin-3-yl}- 2-methylbenzoic acid | Ethyl 5-chloro-1-[2′- hydroxy-3′- (methyloxy)-4- biphenylyl]-3-{[({2- methyl-3- [(methyloxy)carbonyl] phenyl}amino) carbonyl]amino}-1H- pyrrole-2- carboxylate (Intermediate 52) | 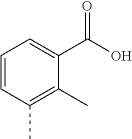 | LCMS: (M + H)⁺ = 518; Rt = 2.66 min. HRMS: calculated for C₂₇H₂₁ClN₃O₆ (M + H)⁺: 518.1119; found: 518.1157 Rt = 2.37 min |
| Example 43 | 3-{6-Chloro-5-[2′- hydroxy-3′- (methyloxy)-4- biphenylyl]-2,4- dioxo-1,2,4,5- tetrahydro-3H- pyrrolo[3,2- d]pyrimidin-3-yl}- 5- (methyloxy) benzoic acid | Ethyl 5-chloro-1-[2′- hydroxy-3′- (methyloxy)-4- biphenylyl]-3-{[({3- (methyloxy)-5- [(methyloxy)carbonyl] phenyl}amino) carbonyl]amino}-1H- pyrrole-2- carboxylate (Intermediate 62) | 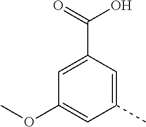 | LCMS: (M + H)⁺ = 534; Rt = 2.56 min. HRMS: calculated for C₂₇H₂₁ClN₃O₇ (M + H)⁺: 534.1068; found: 534.1063 Rt = 2.36 min |
| Example 44 | 6-Chloro-5-[2′- hydroxy-3′- (methyloxy)-4- biphenylyl]-3-[2- methyl-4- (methyloxy)phenyl]- 1H-pyrrolo[3,2- d]pyrimidine- 2,4(3H,5H)-dione | Ethyl 5-chloro-1-[2′- hydroxy-3′- (methyloxy)-4- biphenylyl]-3-[({[2- methyl-4- (methyloxy)phenyl] amino}carbonyl)amino]- 1H-pyrrole-2- carboxylate (Intermediate 55) | 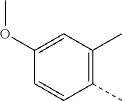 | LCMS: (M + H)⁺ = 504; Rt = 3.38 min. HRMS: calculated for C₂₇H₂₃ClN₃O₅ (M + H)⁺: 504.1326; found: 504.1348 Rt = 2.92 min |
| Example 45 | 6-Chloro-5-[2′- hydroxy-3′- (methyloxy)-4- biphenylyl]-3-(1- naphthalenyl)-1H- pyrrolo[3,2- d]pyrimidine- 2,4(3H,5H)-dione | Ethyl 5-chloro-1-[2′- hydroxy-3′- (methyloxy)-4- biphenylyl]-3-{[(1- naphthalenylamino) carbonyl]amino}- 1H-pyrrole-2- carboxylate (Intermediate 56) | 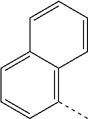 | LCMS: (M + H)⁺ = 510; Rt = 3.31 min. HRMS: calculated for C₂₉H₂₁ClN₃O₄ (M + H)⁺: 510.1220; found: 510.1176 Rt = 2.99 min |
| Example 46 | 3-{6-Chloro-5-[2′- hydroxy-3′- (methyloxy)-4- biphenylyl]-2,4- dioxo-1,2,4,5- tetrahydro-3H- pyrrolo[3,2- d]pyrimidin-3-yl}- 5-methylbenzoic acid | Ethyl 5-chloro-1-[2′- hydroxy-3′- (methyloxy)-4- biphenylyl]-3-{[({3- methyl-5- [(methyloxy)carbonyl] phenyl}amino) carbonyl]amino}-1H- pyrrole-2- carboxylate (Intermediate 63) | 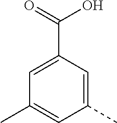 | LCMS: (M + H)⁺ = 518; Rt = 2.74 min. HRMS: calculated for C₂₇H₂₁ClN₃O₆ (M + H)⁺: 518.1119; found: 518.1170 Rt = 2.42 min |

Examples 47 to 49 of formula (I) were prepared by methods analogous to that described for Intermediate 70.

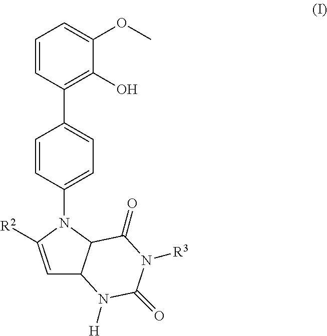

TABLE 14

| | | | | | Physical |
|---|---|---|---|---|---|
| Example | Name | Starting material | R² | R³ | data |
| Example 47 | 3-(2,3- Dimethylphenyl)- 5-[2′- hydroxy-3′- (methyloxy)-4- biphenylyl]- 1H- pyrrolo[3,2- d]pyrimidine- 2,4(3H,5H)- dione | Ethyl 3-({[(2,3- dimethylphenyl) amino]carbonyl} amino)-1-[2′- hydroxy- 3′-(methyloxy)-4- biphenylyl]-1H- pyrrole-2- carboxylate (Intermediate 18) | H | 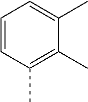 | LCMS: (M + H)⁺ = 454; Rt = 3.22 min. HRMS: calculated for C₂₇H₂₄N₃O₄ (M + H)+: 454.1767; found: 454.1779. Rt: 2.87 min. |
| Example 48 | 3-(3-Chloro-2- methylphenyl)- 5-[2′-hydroxy- 3′- (methyloxy)-4- biphenylyl]- 1H- pyrrolo[3,2- d]pyrimidine- 2,4(3H,5H)- dione | Ethyl 3-({[(3- chloro-2- methylphenyl)amino] carbonyl}amino)- 1-[2′-hydroxy-3′- (methyloxy)-4- biphenylyl]-1H- pyrrole-2- carboxylate (Intermediate 19) | H | 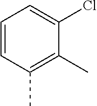 | LCMS: (M + H)⁺ = 474; Rt = 3.31 min. HRMS: calculated for C₂₆H₂₁ClN₃O₄ (M + H)+: 474.1220; found: 474.1227. Rt: 2.96 min. |
| Example 49 | 3-(2- Fluorophenyl)- 5-[2′-hydroxy- 3′- (methyloxy)-4- biphenylyl]- 1H- pyrrolo[3,2- d]pyrimidine- 2,4(3H,5H)- dione | Ethyl 3-({[(2- fluorophenyl)amino] carbonyl}amino)- 1-[2′-hydroxy-3′- (methyloxy)-4- biphenylyl]-1H- pyrrole-2- carboxylate (Intermediate 17) | H | 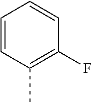 | LCMS: (M + H)⁺ = 444; Rt = 3.09 min. HRMS: calculated for C₂₅H₁₉FN₃O₄ (M + H)+: 444.1360; found: 444.1395. Rt: 2.69 min. |

### Example 50

### 5-(2′-Hydroxy-4-biphenylyl)-1H-pyrrolo[3,2-d]pyrimidine-2,4(3H,5H)-dione

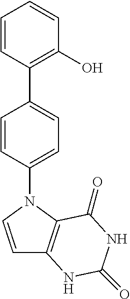 [SMILES: O=c1[nH]c(=O)c2c(ccn2-c2ccc(-c3ccccc3O)cc2)[nH]1]

To a solution 5-(4-bromophenyl)-1H-pyrrolo[3,2-d]pyrimidine-2,4(3H,5H)-dione (Intermediate 57; 200 mg, 0.65 mmol) in 1,4-dioxane (5 mL)/water (3 mL) were added—(2-hydroxyphenyl)boronic acid (108 mg, 0.78 mmol), cesium carbonate (639 mg, 1.96 mmol), and Pd(Ph₃P)₄ (2.27 mg, 1.96 μmol). The reaction vessel was sealed and heated to 160° C. for 20 min in microwave. After cooling the organic layer and was evaporated off. The residue was triturated with MeOH/AcOH (90/10), hot CH₃CN and recrystallised from DMF/H₂O (90/10) to give the title compound 5-(2′-hydroxy-4-biphenylyl)-1H-pyrrolo[3,2-d]pyrimidine-2,4(3H,5H)-dione (25 mg, 0.07 mmol, 11.38% yield) as brown crystals.

LCMS: (M+H)⁺=320; Rt=2.56 min.

HRMS: calculated for C₁₈H₁₂N₃O₃ (M−H)+: 318.0879; found: 318.0860. Rt: 2.28 min.

Examples 51 to 58 of the general formula below were prepared by methods analogous to that described for Example 50 using the appropriate boronic acid.

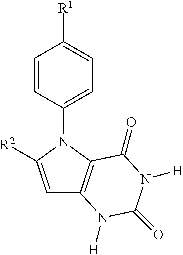

TABLE 15

| | | Starting | | | Physical |
|---|---|---|---|---|---|
| Example | Name | Material | R² | R¹ | data |
| Example 51 | 5-[2′-Hydroxy- 3′- (methyloxy)- 4-biphenylyl]- 1H- pyrrolo[3,2- d]pyrimidine- 2,4(3H,5H)- dione | 5-(4- Bromophenyl)- 1H-pyrrolo[3,2- d]pyrimidine- 2,4(3H,5H)-dione (Intermediate 57) and [2-hydroxy-3- (methyloxy) phenyl]boronic acid | H | 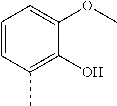 | LCMS: (M + H)⁺ = 350; Rt = 4.88 min. HRMS: calculated for C₁₉H₁₆N₃O₄ (M + H)+: 350.1141; found: 350.1136. Rt: 2.42 min. |
| Example 52 | 5-(3′-Fluoro- 2′-hydroxy-4- biphenylyl)- 1H- pyrrolo[3,2- d]pyrimidine- 2,4(3H,5H)- dione | 5-(4- Bromophenyl)- 1H-pyrrolo[3,2- d]pyrimidine- 2,4(3H,5H)- dione (Intermediate 57) and (3- fluoro-2- hydroxyphenyl) boronic acid | H | 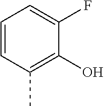 | LCMS: (M + H)⁺ = 338; Rt = 2.46 min. HRMS: calculated for C₁₈H₁₂FN₃O₃ (M − H)+: 336.0785; found: 336.0780. Rt: 2.29 min. |
| Example 53 | 5-(5′-Fluoro- 2′-hydroxy-4- biphenylyl)- 1H- pyrrolo[3,2- d]pyrimidine- 2,4(3H,5H)- dione | 5-(4- Bromophenyl)- 1H-pyrrolo[3,2- d]pyrimidine- 2,4(3H,5H)- dione (Intermediate 57) and (5- fluoro-2- hydroxyphenyl) boronic acid | H | 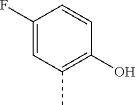 | LCMS: (M + H)⁺ = 338; Rt = 2.49 min. HRMS: calculated for C₁₈H₁₃FN₃O₃ (M + H)+: 338.0941; found: 338.0914. Rt: 2.31 min. |
| Example 54 | 5-(4′-Fluoro- 2′-hydroxy-4- biphenylyl)- 1H- pyrrolo[3,2- d]pyrimidine- 2,4(3H,5H)- dione | 5-(4- Bromophenyl)- 1H-pyrrolo[3,2- d]pyrimidine- 2,4(3H,5H)- dione (Intermediate 57) and (4- fluoro-2- hydroxyphenyl) boronic acid | H | 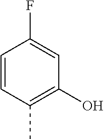 | LCMS: (M + H)⁺ = 338; Rt = 2.52 min. HRMS: calculated for C₁₈H₁₁FN₃O₃ (M − H)+^{_{:}} 336.0785; found: 336.0783. Rt: 2.34 min. |
| Example 55 | 5-(4′-Chloro- 2′-hydroxy-4- biphenylyl)- 1H- pyrrolo[3,2- d]pyrimidine- 2,4(3H,5H)- dione | 5-(4- Bromophenyl)- 1H-pyrrolo[3,2- d]pyrimidine- 2,4(3H,5H)- dione (Intermediate 57) and (4- chloro-2- hydroxyphenyl) boronic acid (Intermediate 67) | H | 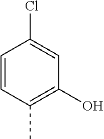 | LCMS: (M + H)⁺ = 354; Rt = 5.46 min. HRMS: calculated for C₁₈H₁₁ClN₃O₃ (M − H)+: 352.0489; found: 352.0483. Rt: 2.50 min. |
| Example 56 | 6-Chloro-5-[2′- hydroxy-3′- (methyloxy)-4- biphenylyl]- 1H- pyrrolo[3,2- d]pyrimidine- 2,4(3H,5H)- dione | 5-(4- Bromophenyl)- 6-chloro-1H- pyrrolo[3,2- d]pyrimidine- 2,4(3H,5H)- dione (Intermediate 59) and [2- hydroxy-3- (methyloxy) phenyl]boronic acid | Cl | 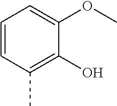 | LCMS: (M − H)⁺ = 382; Rt = 2.77 min. HRMS: calculated for C₁₉H₁₃ClN₃O₄ (M − H)+: 382.0595; found: 382.0611. Rt: 2.47 min. |
| Example 57 | 6-Chloro-5- (2′-hydroxy-4- biphenylyl)- 1H- pyrrolo[3,2- d]pyrimidine- 2,4(3H,5H)- dione | 5-(4- Bromophenyl)- 6-chloro-1H- pyrrolo[3,2- d]pyrimidine- 2,4(3H,5H)- dione (Intermediate 59) and (2- hydroxyphenyl) boronic acid | Cl | 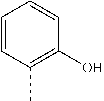 | LCMS: (M − H)⁺ = 352; Rt = 2.68 min. HRMS: calculated for C₁₈H₁₁ClN₃O₃ (M − H)+: 352.0489; found: 352.0478. Rt: 2.42 min. |
| Example 58 | 6-Chloro-5- (2′-hydroxy-3′- methyl-4- biphenylyl)- 1H- pyrrolo[3,2- d]pyrimidine- 2,4(3H,5H)- dione | 5-(4- Bromophenyl)- 6-chloro-1H- pyrrolo[3,2- d]pyrimidine- 2,4(3H,5H)- dione (Intermediate 59) and (2- hydroxy-3- methylphenyl) boronic acid | Cl | 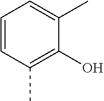 | LCMS: (M + H)⁺ = 368; Rt = 2.95 min. HRMS: calculated for C₁₉H₁₅ClN₃O₃ (M + H)+: 368.0802; found : 368.0804. Rt: 2.70 min. |

### Example 59

### 5-(2′-Fluoro-6′-hydroxy-4-biphenylyl)-1H-pyrrolo[3,2-d]pyrimidine-2,4(3H,5H)-dione

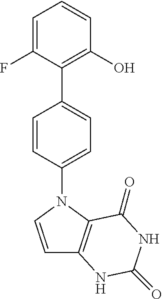 [SMILES: O=c1[nH]c(=O)c2c(ccn2-c2ccc(-c3c(O)cccc3F)cc2)[nH]1]

To a solution of 5-[2′-fluoro-6′-(methyloxy)-4-biphenylyl]-1H-pyrrolo[3,2-d]pyrimidine-2,4(3H,5H)-dione (Intermediate 60; 80 mg, 0.21 mmol) in DCM (5 mL) was added BBr₃ at 0° C. (0.62 mL, 0.62 mmol). The mixture was stirred at RT over 2 days. The mixture was evaporated and 110 mg of a crude product was obtained. The product was purified by chromatography using a DCM/MeOH 100/0 to 90/10 as gradient. The appropriate fractions were combined and concentrated in vacuo to give the title compound 5-(2′-fluoro-6′-hydroxy-4-biphenylyl)-1H-pyrrolo[3,2-d]pyrimidine-2,4(3H,5H)-dione as a white solid after trituration in diethyl ether (10 mg, 0.03 mmol, 90% yield).

LCMS: (M+H)⁺=338; Rt=2.48 min.

HRMS: calculated for C₁₈H₁₃FN₃O₃ (M+H)+: 338.0941; found: 338.0937. Rt: 2.30 min.

### Example 60

### 3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-N-methylpropanamide

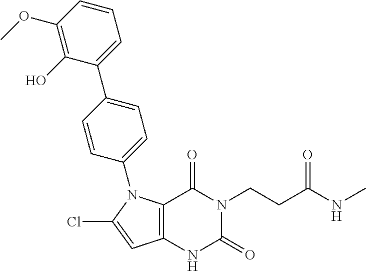

To a suspension of 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid (Example 1) (150 mg, 0.33 mmol) in a mixture of DCM (10 mL) and tetrahydrofuran (10 mL) was added HATU (188 mg, 0.49 mmol) and methylamine was bubbled for 5 min. The reaction was stirred at RT overnight. The insoluble was filtered and the filtrate was evaporated to dryness and was purified by chromatography using DCM/MeOH 95/5 as gradient. Pure fractions were combined and concentrated in vacuo. The compound was precipitated in water, filtered and dried to give the title compound 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-N-methylpropanamide (30 mg, 0.06 mmol, 19.44% yield) as a off white solid.

LCMS: (M+H)⁺=469; Rt=2.87 min.

HRMS: calculated for C₂₃H₂₂ClN₄O₅ (M+H)+: 469.1279; found: 469.1248. Rt: 2.56 min.

### Example 61

### Ethyl 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoate

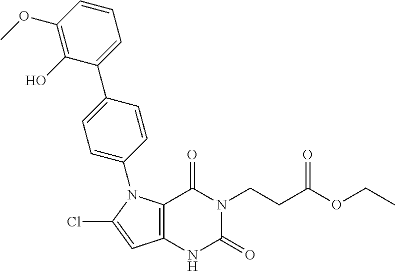 [SMILES: CCOC(=O)CCn1c(=O)[nH]c2cc(Cl)n(-c3ccc(-c4cccc(OC)c4O)cc3)c2c1=O]

To a solution of 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid (370 mg, 0.81 mmol; Example 1) in ethanol (100 mL) was bubbled HCl gas. The reaction was then stirred at 80° C. for 4 hours and concentrated in vacuo. The product was purified by chromatography on silica gel using DCM to DCM/MeOH (90/10) as gradient. The pure fractions was concentrated in vacuo and the residue was dissolved in ethanol (1 mL) and precipitated in water (20 mL). The precipitate was filtered, washed with water and dried to give the title compound, ethyl 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoate (240 mg, 0.50 mmol, 61.1% yield) as a off white powder.

LCMS: (M+H)⁺=484; Rt=3.32 min.

HRMS: calculated for C₂₄H₂₃ClN₃O₆ (M+H)+: 484.1275; found: 484.1259. Rt: 2.88 min.

### Example 62

### 3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}benzoic acid

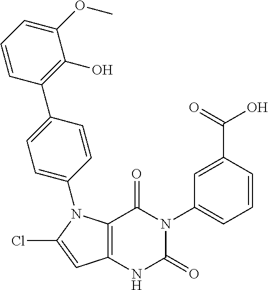 [SMILES: COc1cccc(-c2ccc(-n3c(Cl)cc4[nH]c(=O)n(-c5cccc(C(=O)O)c5)c(=O)c43)cc2)c1O]

To a solution of ethyl 5-chloro-3-{[({3-[(ethyloxy)carbonyl]phenyl}amino)carbonyl]amino}-1-[2′-hydroxy-3′-(methyloxy)-4-biphenylyl]-1H-pyrrole-2-carboxylate (Intermediate 64; 250 mg, 0.43 mmol) in ethanol (20 mL) was added sodium (29.8 mg, 1.3 mmol). The reaction was stirred at 90° C. for 8 hours. After cooling, the reaction was concentrated in vacuo and acidified with a 1N HCl solution. The solid was filtered, washed with water and dried. The product was purified by chromatography on silica gel using DCM to DCM/MeOH (90/10) as gradient. The pure fractions was concentrated in vacuo and the residue was dissolved in ethanol (1 mL) and precipitated in water. The precipitate was filtered, washed with water and dried to give the title compound, 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}benzoic acid (60 mg, 0.12 mmol, 27.5% yield) as a off white solid.

LCMS: (M+H)⁺=504; Rt=2.55 min.

HRMS: calculated for C₂₆H₁₉ClN₃O₆ (M+H)+: 504.0962; found: 504.0929. Rt: 2.35 min.

Example 63 of the general formula below was prepared by methods analogous to that described for Example 62 using the appropriate intermediate.

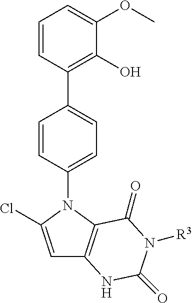

TABLE 16

| Example | Name | Starting Material | R³ | Physical data |
|---|---|---|---|---|
| Example 63 | 4-{6-Chloro-5-[2′- hydroxy-3′- (methyloxy)-4- biphenylyl]-2,4- dioxo-1,2,4,5- tetrahydro-3H- pyrrolo[3,2- d]pyrimidin-3- yl}benzoic acid | Ethyl 5-chloro-3-{[({4- [(ethyloxy)carbonyl] phenyl}amino)carbonyl] amino}-1-[2′- hydroxy-3′- (methyloxy)-4- biphenylyl]-1H- pyrrole-2-carboxylate (Intermediate 65) | 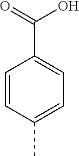 | LCMS: (M + H)⁺ = 504; Rt = 2.65 min. HRMS: calculated for C₂₆H₁₉ClN₃O₆ (M + H)+: 504.0962; found: 504.0955. Rt: 2.35 min. |

### Example 64

### 1-Methylethyl 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoate

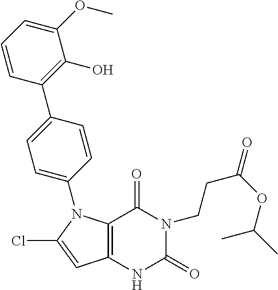 [SMILES: COc1cccc(-c2ccc(-n3c(Cl)cc4[nH]c(=O)n(CCC(=O)OC(C)C)c(=O)c43)cc2)c1O]

To a solution of 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid (420 mg, 0.92 mmol; Example 1) in isopropanol (20 mL) was bubbled hydrochloric acid gas. The reaction was stirred at 80° C. for 4 hours. After cooling, the reaction was concentrated in vacuo. The product was purified by chromatography on silica gel using DCM to DCM/MeOH (90/10) as gradient. The pure fractions were concentrated in vacuo. The precipitate was filtered, washed with EtOH then Et₂O and dried to give the title compound, 1-methylethyl 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoate (450 mg, 0.90 mmol, 98% yield) as a white powder.

LCMS: (M+H)⁺=498; Rt=3.44 min.

HRMS: calculated for C₂₅H₂₅ClN₃O₆ (M+H)⁺: 498.1432; found: 498.1389. Rt: 2.97 min.

### Example 65

### 1-Methylethyl {6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}acetate

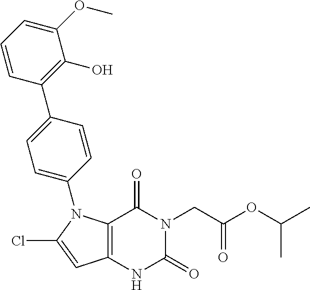 [SMILES: COc1cccc(-c2ccc(-n3c(Cl)cc4[nH]c(=O)n(CC(=O)OC(C)C)c(=O)c43)cc2)c1O]

To a solution of {6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}acetic acid (Example 3; 200 mg, 0.45 mmol) in isopropanol (100 mL) was bubbled hydrogen chloride gas. The reaction was stirred at 80° C. for 4 hours. After cooling, the reaction was concentrated in vacuo. The product was purified by chromatography on silica gel using DCM to DCM/MeOH (90/10) as gradient. The pure fractions were concentrated in vacuo, diluted in ethanol (1 mL) then precipitated in water. The precipitate was filtered, washed with water and dried to give the title compound, 1-methylethyl {6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}acetate (60 mg, 0.12 mmol, 27% yield) as an orange powder.

LCMS: (M+H)⁺=484; Rt=3.40 min.

HRMS: calculated for C₂₄H₂₃ClN₃O₆ (M+H)+: 484.1275; found: 484.1238. Rt: 2.95 min.

Example 66 of the general formula below was prepared by methods analogous to that described for Example 65 using the appropriate starting material.

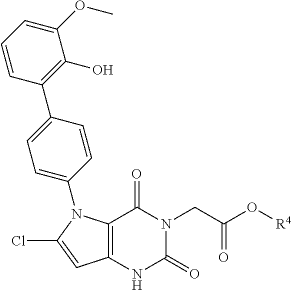

TABLE 17

| | | | | Physical |
|---|---|---|---|---|
| Example | Name | Starting Material | R⁴ | data |
| Example 66 | Ethyl {6-chloro-5-[2′- hydroxy-3′- (methyloxy)-4- biphenylyl]-2,4- dioxo-1,2,4,5- tetrahydro-3H- pyrrolo[3,2- d]pyrimidin-3- yl}acetate | {6-Chloro-5-[2′- hydroxy-3′- (methyloxy)-4- biphenylyl]- 2,4-dioxo- 1,2,4,5-tetrahydro- 3H-pyrrolo[3,2- d]pyrimidin-3- yl}acetic acid (Example 3) | 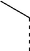 | LCMS: (M + H)⁺ = 470; Rt = 3.15 min. HRMS: calculated for C₂₃H₂₁ClN₃O₆ (M + H)+: 470.1119; found: 470.1127. Rt: 2.84 min. |

### Example 67

### 2-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid

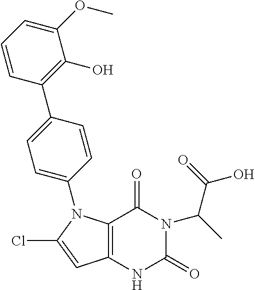 [SMILES: COc1cccc(-c2ccc(-n3c(Cl)cc4[nH]c(=O)n(C(C)C(=O)O)c(=O)c43)cc2)c1O]

To a solution of ethyl 5-chloro-1-[2′-hydroxy-3′-(methyloxy)-4-biphenylyl]-3-[({[1-methyl-2-(methyloxy)-2-oxoethyl]amino}carbonyl)amino]-1H-pyrrole-2-carboxylate (Intermediate 66; 200 mg, 0.39 mmol) in ethanol (20 mL) was added sodium (27 mg, 1.16 mmol). The reaction was stirred at 90° C. for 8 hours. After cooling, the reaction was concentrated in vacuo and acidified with a 1N HCl solution. The precipitate was filtered, washed with water and purified by chromatography on silica gel (reverse phase C18) using H₂O/CH₃CN 10/90 to 50/50 as gradient. The pure fractions were concentrated in vacuo to give the title compound, 2-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid (70 mg, 0.15 mmol, 40% yield) as an off white solid.

LCMS: (M+H)⁺=456; Rt=2.42 min.

HRMS: calculated for C₂₂H₁₉ClN₃O₆ (M+H)+: 456.0962; found: 456.0966. Rt: 2.26 min.

### Biological Assay

### AMPK Enzymatic Assay

Human recombinant AMPK (Invitrogen #PV4673 & #PV4675) is used in a FRET assay format (Z'Lyte—Invitrogen). Assay conditions are as follow: ATP 100 μM, peptide (Invitrogen #PR8650) 2 μM, 1% final DMSO in Z'Lyte kinase buffer. Reaction is initiated by addition of 0.2-0.8 ng of AMPK and incubated for 1-hour @ 30° C. A further 1-hour incubation @ 30° C. with the development reagent (Invitrogen #PR5194) is performed. FRET signal is then measured and converted to “% peptide phosphorylation” according to Z′Lyte given calculation procedure. Evaluation of compounds is carried out using concentration-response curves. Final data are expressed in “% activation” calculating the ratio of “% peptide phosphorylation” between compound-condition and basal-condition. Alternatively pEC200 (−Log(compound concentration leading to a 2-fold AMPK activity increase)) is produced through fitting of the concentration-response curves. All data are means of at least 2 independent experiments.

The compounds of Examples 1 to 67 were tested in the assay described above and gave pEC₅₀ values of greater than 5.5. Certain compounds of the invention give a pEC₅₀ value of ≧6.0 when tested in this assay. Certain compounds of the invention give a pEC₅₀ value of ≧7.0 when tested in this assay.

For instance, Example compounds 24 and 50 gave an average pEC₅₀ value of 5.7 and 5.9 respectively.

The compound: 4-[6-Chloro-5-(2′-hydroxy-3′-methyl-4-biphenylyl)-2,4-dioxo-1,2,4,5-tetrahydro-3H-pyrrolo[3,2-d]pyrimidin-3-yl]benzonitrile gave a pEC₅₀ value of 5.4 when tested in the above assay.

The compound 6-Chloro-3-(3-fluorophenyl)-5-[2′-hydroxy-3′-(methyloxy)-4-biphenylyl]-1H-pyrrolo[3,2-d]pyrimidine-2,4(3H,5H)-dione gave a pEC₅₀ value of 5.3 when tested in the above assay.

The compound 6-chloro-5-(4′-chloro-2′-hydroxy-4-biphenylyl)-1H-pyrrolo[3,2-d]pyrimidine-2,4(3H,5H)-dione gave a pEC₅₀ value of less that 4.5 when tested in the above assay.

1-15. (canceled)

16. A compound of formula (I): 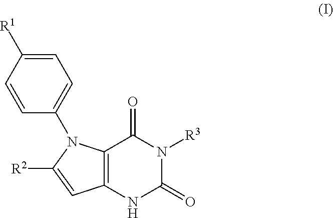
wherein:
R¹ represents —C₆₋₁₀aryl substituted by an —OH group and optionally further substituted by one or two groups independently selected from the group consisting of:
(i) —C₁₋₄alkyl wherein the alkyl group is unsubstituted or substituted by one or two groups independently selected from the group consisting of: —OH and —CO₂H;
(ii) —C₁₋₄alkoxy;
(iii) —OH;
(iv) —CN;
(v) —CO₂H;
(vi) —C₁₋₄haloalkyl;
(vii) —OC₁₋₄haloalkyl;
(viii) —XC(═O)C₁₋₄alkyl; and
(ix) halogen;
R² represents H or halogen;
R³ represents
(a)
(i) —C₁₋₄alkyl wherein the alkyl group is substituted by one or two groups independently selected from the group consisting of: —OH and —CO₂H;
(ii) H;
(iii) —C₁₋₄haloalkyl;
(iv) —C₁₋₄alkyleneOC₁₋₄alkyl; or
(v) —C₁₋₄alkylene(═O)XC₁₋₄alkyl; or
(b) —C₆₋₁₀aryl, wherein the —C₆₋₁₀aryl is unsubstituted or substituted by one or two groups independently selected from the group consisting of:
(i) —C₁₋₄alkyl wherein the alkyl group is unsubstituted or substituted by one or two groups independently selected from the group consisting of: —OH and —CO₂H,
(ii) —C₁₋₄alkoxy;
(iii) —OH;
(iv) —CN;
(v) —NO₂;
(vi) —CO₂H;
(vii) —C₁₋₄haloalkyl;
(viii) —OC₁₋₄haloalkyl;
(ix) —C₁₋₄alkylene(═O)XC₁₋₄alkyl; and
(x) halogen;
X represents O or —NR⁴; and
R⁴ represents H or —C₁₋₄alkyl.

17. The compound of claim 16 wherein R¹ represents phenyl substituted by an —OH group and optionally further substituted by a group independently selected from the group consisting of:
(i) —C₁₋₄alkyl wherein the alkyl group is unsubstituted or substituted by one or two groups independently selected from the group consisting of: —OH and —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —X(═O)C₁₋₄alkyl; and
(ix) halogen.

18. The compound of claim 16 or 17 wherein R² represents H or chloro.

19. The compound of claim 16 wherein R³ represents:
(i) —C₁₋₄alkyl wherein the alkyl group is substituted by one or two groups independently selected from the group consisting of: —OH and —CO₂H;
(ii) H;
(iii) —C₁₋₄haloalkyl;
(iv) —C₁₋₄alkyleneOC₁₋₄alkyl; or
(v) —C₁₋₄alkylene(═O)XC₁₋₄alkyl.

20. The compound of claim 16 wherein:
R¹ represents phenyl substituted by an —OH group and optionally further substituted by a group independently selected from the group consisting of:
(i) —C₁₋₄alkyl wherein the alkyl group is unsubstituted or substituted by one or two groups independently selected from the group consisting of: —OH and —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —X(═O)C₁₋₄alkyl; and
(ix) halogen;
R² represents H or chloro; and
R³ represents:
(i) —C₁₋₄alkyl wherein the alkyl group is substituted by one or two groups independently selected from the group consisting of: —OH and —CO₂H;
(ii) H;
(iii) —C₁₋₄haloalkyl; —C₁₋₄alkyleneO
(iv) C₁₋₄alkyl; or
(v) —C₁₋₄alkylene(═O)XC₁₋₄alkyl.

21. The compound of claim 16 wherein R³ represents —C₆₋₁₀aryl wherein the —C₆₋₁₀aryl is unsubstituted or substituted by one or two groups independently selected from the group consisting of:
(i) —C₁₋₄alkyl wherein the alkyl group is unsubstituted or substituted by one or two groups independently selected from the group consisting of: —OH and —CO₂H,
(ii) —C₁₋₄alkoxy;
(iii) —OH;
(iv) —CN;
(v) —NO₂;
(vi) —CO₂H;
(vii) —C₁₋₄haloalkyl;
(viii) —OC₁₋₄haloalkyl;
(ix) —C₁₋₄alkylene(═O)XC₁₋₄alkyl; and
(x) halogen.

22. The compound of claim 16 wherein:
R¹ represents phenyl substituted by an —OH group and optionally further substituted by a group independently selected from the group consisting of:
(i) —C₁₋₄alkyl wherein the alkyl group is unsubstituted or substituted by one or two groups independently selected from the group consisting of: —OH and —CO₂H;
(ii) —C₁₋₄alkoxy;
(iii) —OH;
(iv) —CN;
(v) —CO₂H;
(vi) —C₁₋₄haloakyl;
(vii) —OC₁₋₄haloalkyl;
(viii) —X(═O)C₁₋₄alkyl; and
(ix) halogen;
R² represents H or chloro; and
R³ represents:
C₆₋₁₀aryl wherein the —C₆₋₁₀aryl is unsubstituted or substituted by one or two groups independently selected from the group consisting of:
(i) —C₁₋₄alkyl wherein the alkyl group is unsubstituted or substituted by one or two groups independently selected from the group consisting of: —OH and —CO₂H,
(ii) —C₁₋₄alkoxy;
(iii) —OH;
(iv) —CN;
(v) —NO₂;
(vi) —CO₂H;
(vii) —C₁₋₄haloalkyl;
(viii) —OC₁₋₄haloalkyl;
(ix) —C₁₋₄alkylene(═O)XC₁₋₄alkyl; and
(x) halogen.

23. The compound of claim 16 wherein the compound is selected from the group consisting of:
3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid;
4-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}butanoic acid;
{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}acetic acid;
3-{5-[2′-Hydroxy-3′-(methyloxy)-4-biphenylyl]-2,4-dioxo-1,2,4,5-tetrahydro-3H-pyrrolo[3,2-d]pyrimidin-3-yl}propanoic acid;
3-{6-Chloro-5-(2′-hydroxy-3′-methyl-4-biphenylyl)-2,4-dioxo-1,2,4,5-tetrahydro-3H-pyrrolo[3,2-d]pyrimidin-3-yl}propanoic acid;
3-[6-Chloro-5-(2′-hydroxy-4-biphenylyl)-2,4-dioxo-1,2,4,5-tetrahydro-3H-pyrrolo[3,2-d]pyrimidin-3-yl]propanoic acid;
6-Chloro-5-[2′-hydroxy-3′-(methyloxy)-4-biphenylyl]-3-phenyl-1H-pyrrolo[3,2-d]pyrimidine-2,4(3H,5H)-dione;
6-Chloro-3-(2,6-dichlorophenyl)-5-[2′-hydroxy-3′-(methyloxy)-4-biphenylyl]-1H-pyrrolo[3,2-d]pyrimidine-2,4(3H,5H)-dione;
6-Chloro-3-(3-chloro-2-fluorophenyl)-5-[2′-hydroxy-3′-(methyloxy)-4-biphenylyl]-1H-pyrrolo[3,2-d]pyrimidine-2,4(3H,5H)-dione;
6-Chloro-3-(3-fluoro-2-methylphenyl)-5-[2′-hydroxy-3′-(methyloxy)-4-biphenylyl]-1H-pyrrolo[3,2-d]pyrimidine-2,4(3H,5H)-dione;
6-Chloro-3-(2,3-dimethylphenyl)-5-[2′-hydroxy-3′-(methyloxy)-4-biphenylyl]-1H-pyrrolo[3,2-d]pyrimidine-2,4(3H,5H)-dione;
3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}benzonitrile;
6-Chloro-3-(3-chloro-2-methylphenyl)-5-[2′-hydroxy-3′-(methyloxy)-4-biphenylyl]-1H-pyrrolo[3,2-d]pyrimidine-2,4(3H,5H)-dione;
6-Chloro-3-(2-chloro-6-methylphenyl)-5-[2′-hydroxy-3′-(methyloxy)-4-biphenylyl]-1H-pyrrolo[3,2-d]pyrimidine-2,4(3H,5H)-dione;
6-Chloro-3-(2,6-difluorophenyl)-5-[2′-hydroxy-3′-(methyloxy)-4-biphenylyl]-1H-pyrrolo[3,2-d]pyrimidine-2,4(3H,5H)-dione;
6-Chloro-5-[2′-hydroxy-3′-(methyloxy)-4-biphenylyl]-3-[3-(trifluoromethyl)phenyl]-1H-pyrrolo[3,2-d]pyrimidine-2,4(3H,5H)-dione;
6-Chloro-3-(2-hydroxyethyl)-5-[2′-hydroxy-3′-(methyloxy)-4-biphenylyl]-1H-pyrrolo[3,2-d]pyrimidine-2,4(3H,5H)-dione;
6-Chloro-5-[2′-hydroxy-3′-(methyloxy)-4-biphenylyl]-3-[2-(methyloxy)ethyl]-1H-pyrrolo[3,2-d]pyrimidine-2,4(3H,5H)-dione;
6-Chloro-5-[2′-hydroxy-3′-(methyloxy)-4-biphenylyl]-3-(2-methylphenyl)-1H-pyrrolo[3,2-d]pyrimidine-2,4(3H,5H)-dione;
6-Chloro-3-(2-chlorophenyl)-5-[2′-hydroxy-3′-(methyloxy)-4-biphenylyl]-1H-pyrrolo[3,2-d]pyrimidine-2,4(3H,5H)-dione;
6-Chloro-3-(3-chlorophenyl)-5-[2′-hydroxy-3′-(methyloxy)-4-biphenylyl]-1H-pyrrolo[3,2-d]pyrimidine-2,4(3H,5H)-dione;
6-chloro-3-(2-fluorophenyl)-5-[2′-hydroxy-3′-(methyloxy)-4-biphenylyl]-1H-pyrrolo[3,2-d]pyrimidine-2,4(3H,5H)-dione;
6-Chloro-5-[2′-hydroxy-3′-(methyloxy)-4-biphenylyl]-3-[3-(methyloxy)phenyl]-1H-pyrrolo[3,2-d]pyrimidine-2,4(3H,5H)-dione;
6-Chloro-5-[2′-hydroxy-3′-(methyloxy)-4-biphenylyl]-3-[4-(methyloxy)phenyl]-1H-pyrrolo[3,2-d]pyrimidine-2,4(3H,5H)-dione;
6-Chloro-3-(4-fluorophenyl)-5-[2′-hydroxy-3′-(methyloxy)-4-biphenylyl]-1H-pyrrolo[3,2-d]pyrimidine-2,4(3H,5H)-dione;
6-Chloro-3-(4-chlorophenyl)-5-[2′-hydroxy-3′-(methyloxy)-4-biphenylyl]-1H-pyrrolo[3,2-d]pyrimidine-2,4(3H,5H)-dione;
6-Chloro-5-[2′-hydroxy-3′-(methyloxy)-4-biphenylyl]-3-(4-methylphenyl)-1H-pyrrolo[3,2-d]pyrimidine-2,4(3H,5H)-dione;
6-Chloro-5-[2′-hydroxy-3′-(methyloxy)-4-biphenylyl]-3-(3-methylphenyl)-1H-pyrrolo[3,2-d]pyrimidine-2,4(3H,5H)-dione;
4-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}benzonitrile;
6-Chloro-5-[2′-hydroxy-3′-(methyloxy)-4-biphenylyl]-3-(2-methyl-6-nitrophenyl)-1H-pyrrolo[3,2-d]pyrimidine-2,4(3H,5H)-dione;
6-Chloro-3-[2-fluoro-3-(trifluoromethyl)phenyl]-5-[2′-hydroxy-3′-(methyloxy)-4-biphenylyl]-1H-pyrrolo[3,2-d]pyrimidine-2,4(3H,5H)-dione;
6-Chloro-3-(2,6-dimethylphenyl)-5-[2′-hydroxy-3′-(methyloxy)-4-biphenylyl]-1H-pyrrolo[3,2-d]pyrimidine-2,4(3H,5H)-dione;
6-Chloro-3-(2,3-dichlorophenyl)-5-[2′-hydroxy-3′-(methyloxy)-4-biphenylyl]-1H-pyrrolo[3,2-d]pyrimidine-2,4(3H,5H)-dione;
6-Chloro-5-[2′-hydroxy-3′-(methyloxy)-4-biphenylyl]-3-(2-methyl-3-nitrophenyl)-1H-pyrrolo[3,2-d]pyrimidine-2,4(3H,5H)-dione;
6-Chloro-5-[2′-hydroxy-3′-(methyloxy)-4-biphenylyl]-3-[3-(methyloxy)propyl]-1H-pyrrolo[3,2-d]pyrimidine-2,4(3H,5H)-dione;
3-[3,4-Bis(methyloxy)phenyl]-6-chloro-5-[2′-hydroxy-3′-(methyloxy)-4-biphenylyl]-1H-pyrrolo[3,2-d]pyrimidine-2,4(3H,5H)-dione;
3-[3,5-Bis(methyloxy)phenyl]-6-chloro-5-[2′-hydroxy-3′-(methyloxy)-4-biphenylyl]-1H-pyrrolo[3,2-d]pyrimidine-2,4(3H,5H)-dione;
6-Chloro-3-(2,4-dimethylphenyl)-5-[2′-hydroxy-3′-(methyloxy)-4-biphenylyl]-1H-pyrrolo[3,2-d]pyrimidine-2,4(3H,5H)-dione;
6-Chloro-5-[2′-hydroxy-3′-(methyloxy)-4-biphenylyl]-3-(2-naphthalenyl)-1H-pyrrolo[3,2-d]pyrimidine-2,4(3H,5H)-dione;
6-Chloro-5-[2′-hydroxy-3′-(methyloxy)-4-biphenylyl]-3-[2-(methyloxy)phenyl]-1H-pyrrolo[3,2-d]pyrimidine-2,4(3H,5H)-dione;
5-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-2-methylbenzoic acid;
3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-2-methylbenzoic acid;
3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-5-(methyloxy)benzoic acid;
6-Chloro-5-[2′-hydroxy-3′-(methyloxy)-4-biphenylyl]-3-[2-methyl-4-(methyloxy)phenyl]-1H-pyrrolo[3,2-d]pyrimidine-2,4(3H,5H)-dione;
6-Chloro-5-[2′-hydroxy-3′-(methyloxy)-4-biphenylyl]-3-(1-naphthalenyl)-1H-pyrrolo[3,2-d]pyrimidine-2,4(3H,5H)-dione;
3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-5-methylbenzoic acid;
3-(2,3-Dimethylphenyl)-5-[2′-hydroxy-3′-(methyloxy)-4-biphenylyl]-1H-pyrrolo[3,2-d]pyrimidine-2,4(3H,5H)-dione;
3-(3-Chloro-2-methylphenyl)-5-[2′-hydroxy-3′-(methyloxy)-4-biphenylyl]-1H-pyrrolo[3,2-d]pyrimidine-2,4(3H,5H)-dione;
3-(2-Fluorophenyl)-5-[2′-hydroxy-3′-(methyloxy)-4-biphenylyl]-1H-pyrrolo[3,2-d]pyrimidine-2,4(3H,5H)-dione;
5-(2′-Hydroxy-4-biphenylyl)-1H-pyrrolo[3,2-d]pyrimidine-2,4(3H,5H)-dione;
5-[2′-Hydroxy-3′-(methyloxy)-4-biphenylyl]-1H-pyrrolo[3,2-d]pyrimidine-2,4(3H,5H)-dione;
5-(3′-Fluoro-2′-hydroxy-4-biphenylyl)-1H-pyrrolo[3,2-d]pyrimidine-2,4(3H,5H)-dione;
5-(5′-Fluoro-2′-hydroxy-4-biphenylyl)-1H-pyrrolo[3,2-d]pyrimidine-2,4(3H,5H)-dione;
5-(4′-Fluoro-2′-hydroxy-4-biphenylyl)-1H-pyrrolo[3,2-d]pyrimidine-2,4(3H,5H)-dione; 5-(4′-Chloro-2′-hydroxy-4-biphenylyl)-1H-pyrrolo[3,2-d]pyrimidine-2,4(3H,5H)-dione;
6-Chloro-5-[2′-hydroxy-3′-(methyloxy)-4-biphenylyl]-1H-pyrrolo[3,2-d]pyrimidine-2,4(3H,5H)-dione;
6-Chloro-5-(2′-hydroxy-4-biphenylyl)-1H-pyrrolo[3,2-d]pyrimidine-2,4(3H,5H)-dione;
6-Chloro-5-(2′-hydroxy-3′-methyl-4-biphenylyl)-1H-pyrrolo[3,2-d]pyrimidine-2,4(3H,5H)-dione;
5-(2′-Fluoro-6′-hydroxy-4-biphenylyl)-1H-pyrrolo[3,2-d]pyrimidine-2,4(3H,5H)-dione;
3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}-N-methylpropanamide;
Ethyl 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoate;
3-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}benzoic acid;
4-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}benzoic acid;
1-Methylethyl 3-{6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoate;
1-Methylethyl {6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}acetate;
Ethyl {6-chloro-5-[2′-hydroxy-3′-(methyloxy)-4-biphenylyl]-2,4-dioxo-1,2,4,5-tetrahydro-3H-pyrrolo[3,2-d]pyrimidin-3-yl}acetate; and
2-{6-Chloro-5-[2′-hydroxy-3′-(methyloxy)-4-biphenylyl]-2,4-dioxo-1,2,4,5-tetrahydro-3H-pyrrolo[3,2-d]pyrimidin-3-yl}propanoic acid.

24. A salt of the compound of claim 16.

25. A pharmaceutical composition comprising a) the compound of claim 16 and b) one or more pharmaceutically acceptable carriers.

26. A pharmaceutical composition comprising a) the salt of claim 24 and b) one or more pharmaceutically acceptable carriers.

27. A method for AMPK activation in a subject comprising administering to said subject the compound of claim 16.

28. A method for the treatment of cancer in a subject in need thereof comprising administering to said subject the compound of claim 16.

29. A method for the prophylaxis of cancer in a subject in need thereof comprising administering to said subject the compound of claim 16.
